(19) United States
(12) Patent Application Publication
(10) Pub. No.: US 2009/0307784 A1
(43) Pub. Date: Dec. 10, 2009

(54) Methods of Analysing Cell Behaviour

(75) Inventor: Philip H. Jones, London (GB)

Correspondence Address: HOWREY LLP - East, C/O IP DOCKETING DEPARTMENT, 2941 FAIRVIEW PARK DR, SUITE 200, FALLS CHURCH, VA, 22042-2924, US

(73) Assignee: Medical Research Council, London (GB)

(21) Appl. No.: 12/090,464

(22) PCT Filed: Oct. 20, 2006
(86) PCT No.: PCT/GB06/03922

§ 371 Date: Apr. 16, 2008

Related U.S. Application Data
(60) Provisional application No. 60/729,032, filed on Oct. 21, 2005.

(30) Foreign Application Priority Data
Oct. 21, 2005 (GB) 0521523.1

(51) Int. Cl. G01N 33/48

(52) U.S. Cl. 800/3

(57) ABSTRACT

The invention related to a method of imaging a clonal cell line comprising providing a test animal comprising a marker gene, inducing inheritable activation of said marker in at least one cell of said test animal, wherein inheritable activation is induced in fewer than 1 in 27 cells in the tissue of interest, incubating the test animal, and visualising those clonal cells which express the marker gene as a result of the inheritable activation. In particular the invention concerns-methods where the tissue is epidermis, and wherein the visualisation is by confocal microscopy such as wholemount confocal microscopy. The invention also relates to toxicity and carcinogenicity testing using such methods.

### FIELD OF THE INVENTION

The invention is in the field of the study of cell behaviour and modelling same. In particular the invention is in the field of modelling cancer and determination of carcinogenicity/toxicity.

### BACKGROUND TO THE INVENTION

Skin mounts a robust recovery on wounding. This recovery is based on the expansion and differentiation of stem cell populations in the skin, including stem cell populations in the inter-follicular epidermis (IFE) as well as stem cell populations associated with the follicles themselves. This regeneration is clinically important from the perspective of recovery from wounding, and also from the perspective of injury to the skin caused in the process of treatment for example following radiological treatments where skin burning and/or skin scorching can occur.

As well as a clinical interest in the regeneration, process, understanding stem cell behaviour is important in modelling cancer.

Stem cells have been identified in the bulge of the hair follicle. A mouse was engineered to express green fluorescent protein throughout its tissues. The green fluorescent protein expression was then turned off. The mouse was then monitored to see which of the cells retained green fluorescent protein the longest. These elegant experiments localised populations of stem cells. However, it was not possible to follow the behaviour of these stem cells such as their pattern of proliferation or differentiation over time. Furthermore, although it is known that stem cells in the bulge can go on to produce hair, skin or sebaceous tissue, it is not known whether bulge stem cells actually support the other pools of stem cells found in the epidermis. The techniques and materials available in the prior art have so far not been able to address this question.

One approach to the study of stem cell function in vivo is double labelling of cycling cells with sequential pulses of tritiated thymidine and bromedeoxyuridine; this shows that proliferating keratinocytes, migrate from the outer root sheath of the hair follicle into the basal layer of the adjacent IFE in neonatal mice and in adult animals following wounding. However, it is not clear whether such migration occurs in uninjured adult epidermis.

Another prior art technique which has been used to try to dissect some of the events in epidermal regeneration is that of retroviral lineage marking. These experiments are essentially wound healing experiments. Unfortunately, it is not possible to trace lineages to individual cells in this style of investigation. Furthermore, it is frequently unclear if clusters, of marked cells are clonal or arise from multiple adjacent infected cells.

Characterisation of epidermal transit amplifying (TA) cells has been limited. Cultured TA cells isolated from human epidermis undergo 2-5 rounds of cell division, after which all of their progeny terminally differentiate, but whether this reflects TA cell behaviour in confluent epidermis is unknown. In retro viral marking and transgenic mouse studies in which the epidermis has been analysed using conventional histological sectioning; it is not possible to detect clusters of 2-32 cells such as would be expected to be produced by TA cells in vivo. Thus, prior art techniques for studying TA cells are problematic.

Thus, despite advances in understanding stem cell/transit amplifying cell behaviour, problems remain. Whilst the label retaining cell approach has been successful in delineating the location of slowly cycling cells, the location of proliferating stem and transit cells and the fate of their progeny cannot be defined by this approach. Although bulge stem cells have the potential to generate upper hair follicle, sebaceous gland and IFE cells, the extent to which they do this in normal adult epidermis is unclear.

It is a problem in the art that there is no satisfactory model of cancer beginning from a one cell (stem cell) stage.

Currently, toxicity testing for carcinogenesis is a very animal intensive process. Relatively large cohorts of test animals such as mice are required to be treated and individually observed for signs of carcinogenesis. These animals clearly come at a large economic and moral cost. It is clearly desirable to reduce the number of animals required for such testing, both to reduce labour and costs of such testing, and to reduce the number of animals sacrificed in these techniques, and to reduce suffering, by requiring fewer animals.

The present invention seeks to overcome problems associated with the prior art.

### SUMMARY OF THE INVENTION

The invention is based on a new combination of a variety of individual techniques.

Overall, the invention involves the careful induction of a recombinant marker inside individual mouse cells. Due to the recombination event which is triggered, this marker becomes an inheritable marker. Therefore, each of the daughter cells generated from the cell harbouring the initial recombination event can be traced individually.

Following the recombination event, the animals, eg. mice, are incubated which allows the various marked cells to undergo their expansion and/or differentiation as appropriate. At particular time points following this incubation stage, the expanded cellular clones are visualised.

Thus, in overview the invention involves selectively triggering recombination events in individual cells within a living mouse. These individual events give rise to traceable visualisable marking of single cells. Over time, these single cells will expand or differentiate dependent on their type and their microenvironment. When the mouse is sacrificed, the proliferative behaviour of the individual cell which was labelled at the outset can be traced back by studying the pattern of the visualised cells.

This new technique advantageously allows a cross section of the whole proliferative process to be seen. In particular, the careful titration of the induction event in order to analyse single cell recombination events (i.e. tagging a single cell and following the events downstream) is highly advantageous.

Optionally, this technique involves the confocal reconstruction of separated epidermis. This method also makes possible quantitation in terms of the amount of epidermis or the populations of stem cells which are being studied. This has not been possible with prior art techniques.

Thus, in one aspect the invention provides a method of imaging a clonal cell line comprising:
(i) providing a test animal comprising a marker gene,
(ii) inducing inheritable activation of said marker in at least one cell of said test animal, wherein inheritable activation is induced in fewer than 1 in 27 cells in the tissue of interest,
(iii) incubating the test animal, and
(iv) visualising those clonal cells which express the marker gene as a result of the inheritable activation.

Preferably the clonal cell line is a single clonal cell line, ie. a clonal cell line arising from a single cell. Clearly, following division and/or differentiation there will typically be numerous cells which derive from the initial single cell in which the recombination event took place. Due to differentiation, these cells may no longer be identical in the traditional sense of a clonal cell line. Here, the term ‘clonal cell line’ refers to the derivation of the cells from a single cell, even if they subsequently undergo differentiation and can be told apart (eg. morphologically or by profiling of gene expression) thereafter. Preferably clonal cells are delineated as those sharing expression of the marker as a result of the recombination event. Preferably clonal cells are those which have descended from a common ancestor cell in which recombination was induced.

Prior art techniques have not permitted the visualisation of clonal cells/clonal cell lines. Prior art techniques have been based on whole tissue X-gal staining which leaches and permeates the tissue rather than being associated with individual cells-expressing the marker gene. Prior art techniques have not enabled the tracing of individual cells derived from a common ancestor since the only wholemount techniques employed which could theoretically cover enough tissue have been crude low-resolution analyses which have served the purposes of the prior art investigations. There has been no need and no motivation in the art to go beyond low-resolution imaging such as. dissecting microscope imaging. Furthermore, this would be impossible in prior art settings such as the gut since the cells of interest are underlain by opaque tissue layers and thus in any case cannot be analysed as taught by the present inventors. A key advantage of the present invention is the capacity to analyse single clonal cells/clonal cell lines which has not been possible in the prior art.

Preferably the clonal cell line is in-vivo ie. within the test animal.

Preferably recombination means a single recombination event producing expression of the marker. ‘Single recombination event’ should not be taken to literally mean a single nucleic acid cleavage and religation. This phrase is used to describe the molecular events associated with a cell undergoing the recombination leading to expression and the marker gene. Preferably the recombination is somatic recombination and preferably the single recombination event is a single somatic recombination event.

Expression of the marker is preferably stable. ‘Stable’ means permanent or persistent throughout the remaining life of that cell. Preferably the expression is constitutive. Constitutive does not always equate with stable since if the marker is under the control of a promoter which activity varies, (eg. varies with the cell cycle) then clearly the resulting expression would not be constitutive but would still be stable in the sense that it requires no further recombination/transduction/transfection events to maintain it following the induced recombination event.

Expression of the marker must be heritable once induced by recombination. In this context, heritable means that the cell will pass on the expression to its daughters. Heritable in this context does not always mean inherited by reproduction of the test animal since as will be apparent to a skilled reader, the invention is primarily focused on somatic mutations rather than germ line mutations. Thus, preferably heritable means inherited by the products of cell division from the cell in which the recombination event took place.

In particular, the expression ‘single recombination event’ is used to refer to the level of induction of recombination at which it is statistically unlikely that neighbouring cells will each, undergo recombination. The level of induction leading to a ‘single recombination event’ should be sufficiently low that individual cells undergoing recombination leading to expression of the marker gene can be spatially distinguished from one another. Thus, a level of induction which led to such a high proportion of induction that neighbouring cells would be likely to both undergo recombination would NOT be considered to be a level of induction of a ‘single recombination event’. For example, in the context of epidermal systems, if induction leads to recombination of more than 1 in 27 cells of a given type then it would no longer be considered to be reliably inducing single recombination events in the sense of the present invention since the chance of neighbouring cells both recombining would be too high. Of course an understanding of the laws of probability means that any level of recombination, however low, can theoretically lead to the possibility of neighbouring cells recombining independently. However, for the purposes of the present invention, the above limit will be taken to indicate the highest proportion of cells induced which would be considered acceptable for the study of single recombination events according to the present invention.

Thus, preferably induction of recombination is at a level that leads to induction of recombination in fewer than 1 in 27 cells, preferably fewer than 1 in 30 cells, preferably fewer than 1 in 40 cells, preferably fewer than 1 in 60 cells, preferably fewer than 1 in 100 cells, preferably fewer than 1 in 150 cells, preferably fewer than 1in 200 cells, preferably-fewer than 1 in 300 cells, preferably fewer than 1 in 400 cells, preferably fewer than 1 in 500 cells, preferably fewer than 1 in 600 cells, preferably fewer than 1 in 635 cells, preferably fewer than 1 in 653 cells, preferably fewer than 1 in 700 cells, preferably fewer than 1 in 800 cells, preferably fewer than 1 in 900 cells, preferably fewer than 1 in 1000 cells, or even fewer.

The technical benefit to the specific levels of recombination quoted is that the probability of the cells being spatially separated is maximised. Naturally these figures represent a compromise between the desirability of having numerous clones per animal to minimise the number of animals required, and the need to arrange the level of recombination to be sufficiently low that the changes of inducing recombination events in neighbouring cells is correspondingly low and therefore individual clones can be generated arising from single cells (rather than from a mosaic of neighbouring cells which each underwent recombination). Thus, in choosing the optimum recombination frequency (and thus the optimum induction of recombination) the operator will pay attention to these factors. It will be apparent that the optimum rates of recombination will vary from tissue to tissue depending, upon the cellular makeup and cell spacing which varies from tissue to tissue.

For example, exemplary values for different applications include induction at 1 in 635 or fewer basal cells is preferred in epidermis such as the IFE; induction at 1 in 27 or fewer cells is preferred for outer root sheath cells in the upper hair follicle and induction at 1 in 35 or fewer cells is preferred in the sebaceous glands. The selection of particular induction rates is a matter for the operator with reference to the guidance given herein.

Preferably the methods of the invention are not methods of treatment or diagnosis of a human or animal. Preferably the test animals are non-human animals. Preferably the test animals are mice.

Preferably the marker gene is introduced into the Rosa locus.

Preferably the tissue of interest is skin, preferably epidermis. This has the advantage that it is amenable to confocal imaging. Skin/epidermis is advantageously translucent. Thus, the laser light used in confocal imaging can penetrate the tissue and allow 3-D imaging permitting tracing of cells derived from a single clone. Thus, in another aspect, the invention provides a method as described above wherein the tissue is epidermis.

Preferably the skin is back skin or tail skin. When the test animal has a tail, preferably the tissue is tail skin. This has the advantage of being, more tractable. Furthermore, it has the advantage of a more tightly defined pattern of hair spacing which allows more reproducible analysis. In addition, the quantitative aspects of the invention are advantageously applied to tail skin, preferably quantitative wholemount analysis is applied to tail skin, preferably mouse tail skin. There are also numerous practical advantages to tail skin such as ease of sampling, ease of handling and so on.

In another aspect, the invention provides a method as described above wherein the visualisation is by confocal microscopy. Preferably the visualisation is by wholemount confocal microscopy.

Confocal microscopy such as wholemount confocal microscopy has the advantage that it permits the tracing of cells arising from a single clone. This is in contrast to alternative techniques such as conventional sectioning which suffer from practical problems such as cutting and sample preparation from frozen material. Furthermore, typical conventional sections are approx. 100 μm across and clonal lines will cross section boundaries, preventing meaningful analysis of single cell clones. Moreover, such sections can be physically uncuttable, and suffer from fragility preventing a robust analysis taking place. Use of wholemounts solves at least these problems. Furthermore, it enables non-recombinant cells to be visualised and gives contextual information to the analysis.

Preferably wholemount imaging is applied to epidermal cells. Application of this technique to other cells such as gut can lead to opaque mounts and obscure analysis. This combination of wholemount with epidermal tissue is particularly advantageous for these reasons. Furthermore, layers of cells are more easily separated from epidermis than from other tissues such as gut which suffer from the problem of inseparable opaque layers.

In another aspect, the invention provides a method as described above wherein inducing inheritable activation is performed by inducing recombination in order to produce expression of said marker.

Preferably the recombination is induced by administration of B-napthoflavone and tamoxifen.

Preferably the marker is enhanced yellow fluorescent protein.

Preferably the recombination system is based on cre-lox.

In another aspect, the invention provides a method according to any preceding claim wherein the mouse is AhcreER^{T} and the induction of recombination is carried out by administration of B-napthoflavone together with tamoxifen.

In another aspect, the invention provides a method of assessing the toxicity of a substance or composition comprising imaging as described above a clonal cell line which has been incubated in the presence of said compound or composition.

In another aspect, the invention provides a method of assessing the carcinogenicity of a substance or composition comprising imaging as described above a clonal cell line which has been incubated in the presence of said compound or composition.

In another aspect, the invention provides a method as described above further comprising comparing the images of the clonal cell line incubated in the presence of said substance or composition with the characteristics of a corresponding clonal cell line which has not been incubated in the presence of said substance or composition.

The presence of the substance or composition may be by injection of the test animal, or by other means of systemic introduction into the test animal such as oral administration, or may be topical application eg. by ‘painting’ or otherwise locally administering the substance or composition. In a preferred embodiment, the test animal is a mouse and administration is by topical application to the tail skin, preferably to the exterior of said skin.

The substance may be a compound or may be a mixture of compounds or may be a gene product. When the substance is a gene product, preferably this is delivered by induction of expression within the clonal cells being studied such as by coupling its expression to expression of the marker gene(s) used. Preferred genes to be used in this manner include p53, or other oncogene(s) or candidate oncogene(s) to be analysed;

Currently, a wide variety of chemicals for. use in food or cosmetic injuries have to be tested in animals to determine their toxicity, or their status as carcinogens. This involves the sacrifice and occasionally the suffering, of significant numbers of test animals worldwide. This is clearly undesirable. One key application of the present invention is the generation of mice which individually comprise numerous marked clonal cell lines. For example, by titrating the induction of the recombination event, several hundred individual physically separate clones can be created in the epidermis of a single mouse. Advantageously, each of these individual separable clones can be treated as a data point in toxicity or mutagen testing. In this way, as few as three mice could be employed per compound to be tested. This is because for example approximately 300 or more clones per mouse can be generated using the techniques of the present invention. This advantageously represents an enormous saving in animal numbers in order to meet statutory toxicity or carcinogen testing requirements, since each individual clone on the mouse can be treated as an individual data point, thereby drastically reducing the number of individual animals needed to be sacrificed in order to form the same quality of toxicology or carcinogen report.

In order to follow the fate of stem and TA cells in normal epidermis in vivo we have used low-frequency genetic marking, mediated by inducible ere recombinase to label single cells in the upper hair follicle, interfolliclular epidermis and sebaceous gland. By using confocal microscopy to image wholemounts of epidermis we have been able to follow the fate of both stem and TA cells and their progeny over a 12 month period in vivo.

In another aspect, the invention provides use of a test animal comprising a marker gene, which marker gene is capable of being induced to be inheritably activated in at least one cell of said test animal, in the monitoring of clonal cell lines. Preferably said test animal is a mouse. Preferably said test animal is a mouse comprising AhcreER^{T}. Thus preferably the invention relates to use of a mouse comprising AhcreER^{T} in the monitoring of clonal cell lines.

As explained herein, a clonal cell line in this context means a population of cells which derive from a common ancestor, or which appear to derive from a common ancestor, in which ancestor a single recombination event occurred to inheritably activate the genetic marker. As a default, cells will be regarded as derived from a common ancestor if they are spatially clustered consistent with this and if they each express the activated marker even if the theroretical possibility of the clonal line arising from a chance occurrence of a plurality of recombination events in neighbouring cells cannot be experimentally excluded. Preferably the cells of the clonal line each derive from a single common ancestor.

Monitoring of clonal cell lines means observing their status in terms of behaviour, development, proliferation, migration, pattern of division, or other characteristics. Preferably monitoring the cell(s) means visualising them, preferably by confocal wholemount imaging. As will be apparent from this document, monitoring cells generally means fixing them and mounting them and therefore the cells are unlikely to undergo further growth, division, differentiation, migration or other events following visualisation. Thus, in aspects of the invention involving cell migration, movement, expansion or other dynamic events, then cohort studies are preferably employed as analysis of individual test animals generally only provides snapshots of the cells in that animal at that time.

In another aspect, the invention provides use of a mouse comprising AhcreER^{T} in the monitoring of expansion or differentiation of at least one cell arising from a single somatic recombination event.

In another aspect, the invention provides a use as described above wherein a single clonal cell line is monitored.

In another aspect, the invention provides use of a cohort of test animals, wherein at least one single recombination event is induced in each animal of the cohort at a starting time point, wherein cells in a first animal of the cohort are examined at a first time point, and cells in a second or further animal of the cohort are examined at second or further time points thereafter. Preferably the test animals are mice. Preferably said mice comprise AhcreER^{T}.

As noted above, cohort analysis, generally involves sacrifice of individual animals at different time points so that a clone of cells analysed at an early time point cannot undergo further incubation (ie. division, expansion etc.) once it has been imaged. Thus, in order to build up a picture of the behaviour of the cells over the time course of the experiment, clones of cells in different animals are analysed at the different time points and the resulting images are collated to provide a reconstruction of the behaviour of a ‘single clone’ over the time of the experiment, each timepoint essentially being a snapshot of the situation at that particular stage. Thus, in this embodiment of the invention it will be apparent that a single clone is regarded as a clone generated at the same induction timepoint but which will be compiled from images collected from comparable clones physically located in different animals at subsequent time points. As will be apparent to the skilled reader, embodiments of the invention relating to analysis of clones over time must be interpreted in this context.

In another aspect, the invention provides use(s) as described above wherein said mouse or mice further comprise R26^{EYFP/EYFP}.

### DETAILED DESCRIPTION OF THE INVENTION

### Epidermal Stem Cells

Epithelia are constantly turned over throughout adult life. In the human epidermis, which consists of layers of keratinocytes, the outermost layer of cells is lost every day. To replace the lost cells, the epidermis contains stem cells, which retain the ability to proliferate and generate new keratinocytes throughout life. In vitro studies with cultured human keratinocytes and in vivo studies in mice indicate that stem cells reside in the basal layer of the interfollicular epidermis (IFE) and in a region of the hair follicle known as the bulge. Bulge stem cells can generate the multiple cell lineages which comprise the hair follicle, whist interfollicular stem cells (IFSC) normally only differentiate into keratinocytes. On average, each stem cell division results in a cell that remains a stem cell and a cell that will differentiate, known as a transit amplifying cell. Evidence from cultured primary keratinocytes, together with BrdU and tritiated thymidine labelling studies in vivo, suggests that the transit amplifying cells undergo several rounds of cell division. After this, all transit amplifying cell progeny terminally differentiate, exiting the cell cycle and migrating from the basal layer of the epidermis, ultimately to be shed from the epidermal surface.

The process of stem cell division is exquisitely regulated so that the number of new keratinocytes generated by stem and transit cells exactly matches the rate of cell loss. Multiple cell signalling pathways, including integrins, hedgehog, wnt and Notch control stem cell behaviour. Disruption of these pathways alters the lineage selection of bulge stem cells and can alter the balance of differentiation and self renewal in cultured human IFSC, leading to excessive production of stem cells or depletion of the stem cell population.

When cancer develops in epithelia, individual stem cells are thought to form expanded clones, spread to form areas of intraepithelial neoplasia and then invade, with additional mutations accompanying each step in transformation. However, this model does not fit the epidemiology of human cancers, and it may be that cancer does not arise from stem cells, but rather from their daughters. Once epidermal progenitors express the first oncogenic mutation, they are thought to acquire further mutations which enable them to escape from the regulatory control imposed by surrounding wild type cells, acquiring further mutations which lead to the development of carcinoma in situ and ultimately invasive cancer. However, to date it has not been possible to test these hypotheses; to do this requires a system to track the behaviour of mutant clones from the single cells carrying an oncogenic mutation into tumours. Understanding pre cancer development is essential for development of cancer preventative drugs and to better define high risk groups for cancer screening. The present invention advantageously provides methods for tracking and imaging clonal cell populations arising from a single cell such as a stem cell/transit amplifying cell.

### Quantitation

In order to exploit the invention for quantitative analysis, three elements are essential. The first of these is a marker system, preferably a fluorescent marker system. The second of these is a confocal imaging protocol. The third of these is a whole mount analysis preferably a whole mount epidermal analysis. It is the combination of these three elements which allows a quantitative readout to be produced, which is advantageous compared to prior art techniques.

### Recombination

An inducible recombination system is a key element of the present invention. For the broadest application a heritable somatic recombination system must be used. The system should be tightly regulated so that no background recombination events, or no significant background recombination events, are observed. In this way, the recombination induction can be carefully titrated so that it occurs at a sufficiently low frequency to allow single cell events to be monitored. If the frequency is too high, then the risk of neighbouring cells both undergoing a recombination event is heightened. This can confound the analysis of the eventual visualised clonal cell lines. However, according to the present invention, the induction is carefully titrated to ensure that on average individual recombination events occur in cells which are sufficiently spatially separated to allow the daughter cells from each of the individual cells to be followed without the physical expansion of the clones causing a merging or demerging of the individual marked populations. In this way, multiple meaningful clones can be analysed for each animal, advantageously reducing the number of animals needed to be sacrificed in any given experiment.

Preferred recombination systems according to the present invention are the cre-lox recombinase or flp recombinase systems. An inducible flp system may be used. In particular, the cre-lox system is preferred, preferably an inducible cre-lox system.

Particularly preferred is the AhcreER^{T} system (Kemp et al, 2004 NAR-vol 32 No. 11).

Any similar drug induced systems may be used, for example based on cytochrome promoters. Another possible route would be to apply/inject ere recombinase protein to the tissue of interest, preferably skin.

A recombination system for use in the present invention preferably meets the following criteria:

Recombination efficiency proportionate to inducer dose (such as inducing drug dose), so recombination frequency can be adjusted to an appropriate level to visualise individual clones.

No background recombination in the absence of inducer.

These criteria are met by AhcreER^{T}. Thus, preferably the recombination system of the present invention is AhcreER^{T}.

### Recombination Locus

The genetic construct such as the marker gene is directed into a particular locus of the test animal's genome.

Preferably a ubiquitously expressed locus is used in the present invention. Preferably the conditional cassette is targeted to the hprt or Rosa-locus, preferably the Rosa-locus. Furthermore, the expression of a gene of interest such as an oncogene can be restricted by using a tissue specific promoter, such as keratin 5 which directs expression to the basal layer of the epidermis.

### Incubation

By incubation we mean incubation of the mouse. The mouse comprises the individual marked clonal cell lines, and so by incubating the mouse the individual clonal lines are also being incubated. Essentially, the clonal lines can be thought of as being incubated in vivo in the tissue of the mouse in which, they were generated. However, clearly the incubation overall (i.e. the mouse) takes place in vitro in a suitable laboratory setting.

The incubation step is intended to allow the normal processes for cell division, migration or differentiation to take place. Thus, mice should be given their normal levels of care and their normal diet and as far as possible normal conditions during the incubation stage. The cells may then expand (or not expand) as they normally would in the particular micro-environment in which they find themselves within the mouse. This is important since it allows the biologically relevant in vivo processes to be dissected according to the present invention.

### Visualisation

Visualisation may be by any suitable means known to the person skilled in the art. For example, a marker may be used which is later detected by an antibody, the antibody mediating the visualisation. Alternatively, the marker may itself be fluorescent. Most preferred are markers which are themselves fluorescent. In highly preferred embodiments, enhanced yellow fluorescent protein is the marker.

It should be noted that simply because the marker is itself fluorescent, it is still perfectly acceptable to use an antibody related visualisation system to detect it. For example, it may be advantageous to use antibody to yellow fluorescent protein in order to visualise it, basing the visualisation on the antibody rather than the inherent fluorescence of the enhanced yellow fluorescent protein. Indeed, this is particularly advantageous for analyses at early time points when signal levels or protein volumes can be quite low.

Preferred detection systems include fluorescent proteins, proteins expressing an epitope tag, allowing visualisation with anti-tag immunoflourescence, proteins which are themselves immunogenic and can be visualised by immunoflourescence, eg mutant p53.

Fluorescent and/or tagged proteins can be expressed from the same RNA as the gene of interest by using an IRES sequence or as a fusion protein with the gene of interest.

Alternatively, fluorescent and/or tagged proteins can be included in a loxP flanked STOP cassette, so that clones are identified by loss of the fluorescent or tagged protein.

Advantageously these complementary approaches can be combined eg. by including a blue fluorescent protein in the STOP cassette, and a yellow fluorescent protein expressed from an IRES with the gene of interest. In this embodiment, following recombination the cells would convert from blue to yellow.

Confocal imaging is preferred for visualising the sections.

Whole mount tissue is preferred for visualising the tissues. Preferably the wholemounts are prepared and treated as in Braun et al. 2003 (Development and Disease vol 130 pp 5241-5255).

### Test Animals

Preferably test animals are non-lumen mammals, preferably test animals are mice.

Mouse strain FVBN is a preferred mouse strain according to the present invention. GLI1 in a C57B6129 background is a preferred mouse system for analysing tumourigenesis. This mouse is prone to the rapid development of tumours.

E67 mice are preferred for the study of early stage lesions, but these mice do not tend to efficiently develop tumours.

When selecting a test animal such as a particular mouse strain, the choice depends on the gene to be studied. For example, considering choice of mouse strain, HPV e6/7 requires an FVB/n background to develop tumours. Gli-1 or 2 transgenics develop tumours in a mixed C57B16/SV129 background.

Generally, “straight” transgenic mice are less desirable for use in the methods of the present invention due to the high levels of variation which can be observed. However, for some embodiments, it may be desirable to use such mice. Overall however a knock-in strategy is highly preferred for the generation of test mammals such as mice according to the present invention.

It is an essential feature of the present invention that the reporter gene (i.e. marker gene) must be linked to the recombination event.

It is an advantage of the present invention that there is substantially zero background recombination before induction. This is a feature of the selection of mouse and construct combinations in accordance with the present invention. Preferred mouse and construct combinations are disclosed herein. However, it will be apparent to the person skilled in the art that it is straightforward to screen other mouse and construct combinations for an advantageous zero background level of recombination. For example, this is believed to depend on the location of the Ah promoter in the genome. A person wishing to generate alternative mice for use in the present invention could simply introduce the All promoter into the genome and screen those mice for zero background recombination events.

### Titration

Titration of the induction of recombination is a key feature of the present invention. It is this careful titration which allows the induction to be carried out at such a low level to enable single cell clonal recombinants to be generated. In the present invention, Rosa is the locus of choice. With reference to the example section, the dosage for the genetic constructs placed in the Rosa locus can be used as an excellent starting position for induction or recombination events when other loci are used. However, it will be important to perform preliminary induction studies when using other loci in order to correctly determine the right level of induction in order to obtain the desired frequency of recombination. This is well within the ability of the skilled reader in the context of this disclosure. For example, when using the construct inserted into a non-Rosa locus, it would be straightforward to follow the procedure using the Rosa induction protocol, and then to increase or decrease the level of induction as appropriate for the alternative loci used.

### INDUSTRIAL APPLICATION

The invention finds application in modelling of cancer, in study of cellular processes and cellular expansion and/or differentiation. Preferably such study is in vivo in a test animal, said animal being studied in vitro.

The invention finds application in toxicity and/or carcinogenesis studies. These studies are often statutory requirements before bringing compounds or compositions to market eg. cosmetic or therapeutic compositions. Furthermore, often such tests are needed to advance the process of drug discovery and/or testing for example before proceeding to full scale clinical trial.

It is recognised that the invention relates to the manipulation and study of experimental animals. However, the methods of the invention do not cause suffering or pain to the animals. Furthermore, although some applications of the invention are in the field of toxicity and/or carcinogen testing, which may cause some discomfort to the animals, it will be appreciated by the reader that the invention allows far fewer animals to be used in such testing than is the case with the prior art. Thus, individual animals will suffer no more than prior art animals, but advantageously according to the present invention dramatically fewer animals may be needed to provide the same amount of toxicity/carcinogen profiling data. Thus, it is clear that overall the present invention is morally desirable since it causes no greater suffering to any individual animal than is already necessary, and advantageously greatly reduces the number of animals needed in test procedures.

### Further Applications and Advantages

When applying the invention to carcinogen testing, preferably clones are engineered which express p53. p53 mutant mice are preferably used for carcinogen testing. In this embodiment, drugs or treatments that alter the fate of p53 mutant clones may be carcinogenic.

Quantitative modelling can be implemented by simple modelling that giving a high quality of fit to several independent data sets derived from the clonal model of the present invention. This enables investigation of the effects of topically applied drugs/clonally expressed genes on clonal fate at early time points (eg. 2 and 3 weeks) and advantageously avoids the need to perform prolonged time courses.

Epidermis is the tissue of choice for carcinogenesis studies and has been the industry standard since at least the 1930s. Epidermis/skin provides an excellent model for skin cancer, and indeed models other disorders such as cervical cancer and cancers of the oesophagus, as well as cancers of any other stratified squamous tissue.

It is an advantage of the invention that cell clones can be followed for up to a year or even more.

It is an advantage of the invention that every cell in the clone(s) can be resolved In a preferred aspect, the invention relates to the combination of controlled low-level induction of recombination with wholemount imaging. It is this combination which permits the visualisation of clones arising from single cell activation (recombination/induction) events.

In one embodiment the invention relates to a clonal model of basal cell carcinoma. In one embodiment this relates to an in vivo system for study of the clonal evolution of cancer, from single, progenitor cells expressing an oncogenic mutation into tumours. In another embodiment this relates to a clonal model of squamous carcinoma using a conditional human papilloma virus E6/E7 transgenic mouse.

The present invention will now be described, by way of example only, in which reference will be made to the following figures:

### BRIEF DESCRIPTION OF THE FIGURES

FIG. 1 shows stem cells in the epidermis.

A: Organisation of the epidermis. Hair follicles contain multilineage stem cells located in the bulge (b, blue). These stem cells have the potential to generate lower hair follicle (If), sebaceous gland (sg, orange) upper follicle (hf) and interfollicular epidermis (IFE), as shown by the arrows. Inset shows the organisation of IFE. Proliferation is confined to the basal layer, which also contains self renewing stem cells (S, blue), together with transit amplifying cells (TA, green). TA cells generate post mitotic basal cells (red), which leave the basal layer and are ultimately shed from the epidermal surface (arrows).

B: Experimental Design. R26^{EYFP/EYFP} mice; with a conditional EYFP (yellow) expression construct containing a “stop” cassette (red) flanked by LoxP sequences (Blue triangles) targeted to the ubiquitous Rosa 26 promoter, were crossed with the Ahcre^{ERT} transgenic strain that expresses cre recombinase fused to a mutant oestrogen receptor (cre^{ERT}) following treatment with βnapthoflavone (βNF) which induces the Ah promoter. In the presence of Tamoxifen, cre^{ERT} mediates excision of the stop cassette resulting in EYFP expression in the recombinant cell and its progeny.

C,D Wholemount imaging of tail epidermis of uninduced Ahcre^{ERT} R26^{EYFP/wt} mice, using confocal microscopy, prior to induction. Cartoons show the angle of view. C, hair follicle, viewed from the basal surface of the epidermis, with regions labelled as in A. Dotted white lines show the boundaries of the upper hair follicle (uf), scale bar represents 50 μm. D, low power view of the basal surface of tail epidermis, scale bar represents 200 μm. Red dotted line shows the unit area of IFE described in the text;

E,F cartoons showing boundaries of IFE unit area, E is a view of the basal surface, F, a lateral view.

FIG. 2 shows inducible clonal marking: EYFP expressing clones in the IFE over 1 year following induction.

A: Projected Z stack confocal images of interfollicular epidermal wholemounts from Ahcre^{ERT} R26^{EYFP/wt} mice viewed from the basal epidermal surface at the time points shown over a 1 year time course following induction. Cartoon indicates angle of view. Yellow, EYFP; blue, DAPI nuclear stain. Scale bar represents 20 μm.

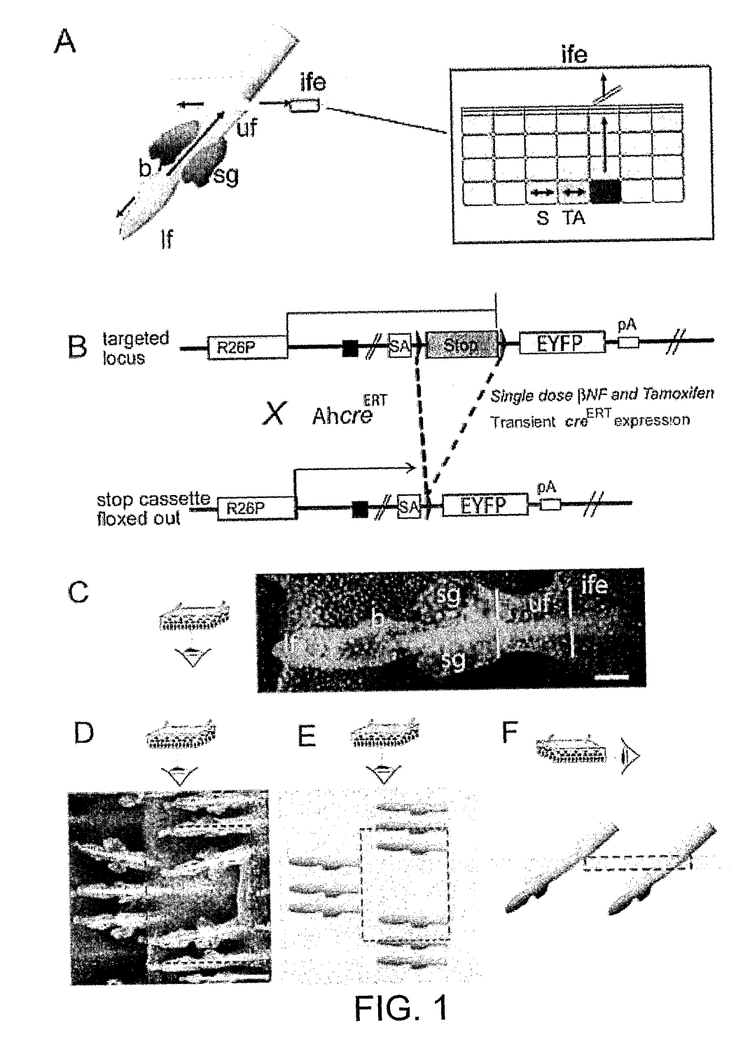

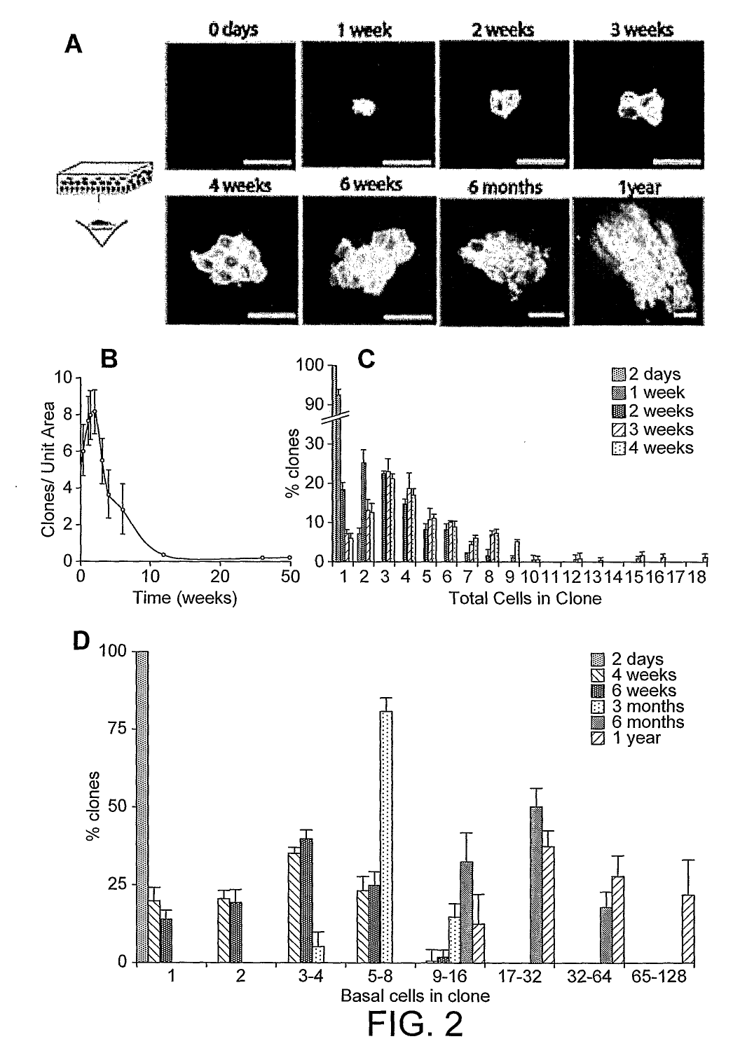

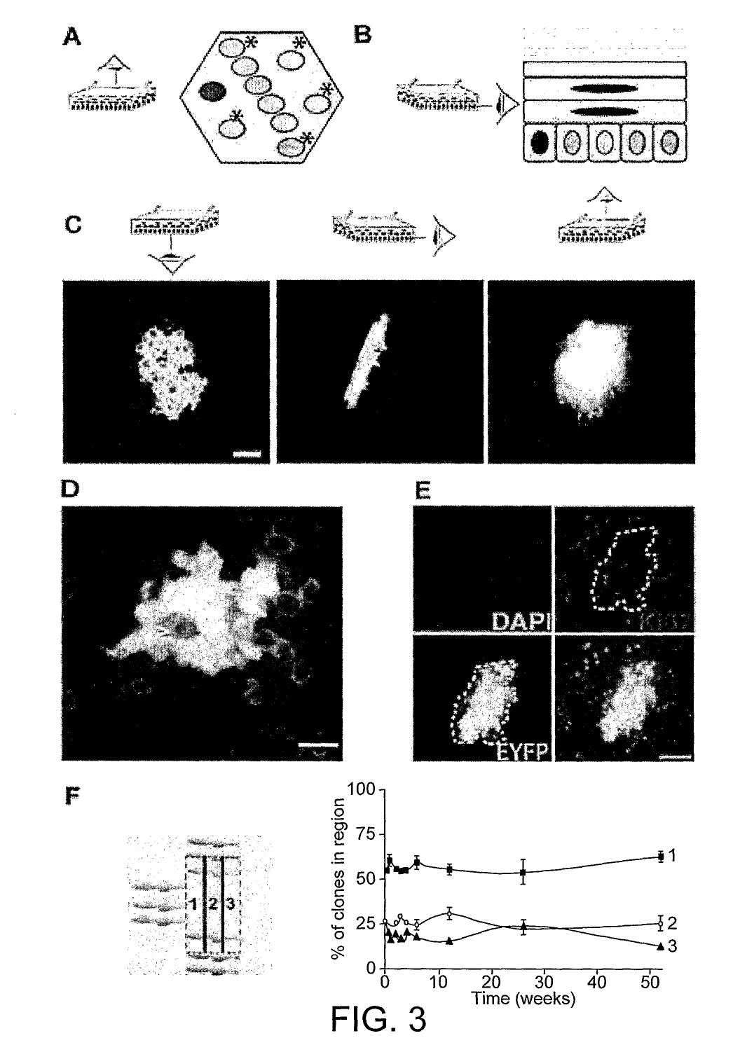

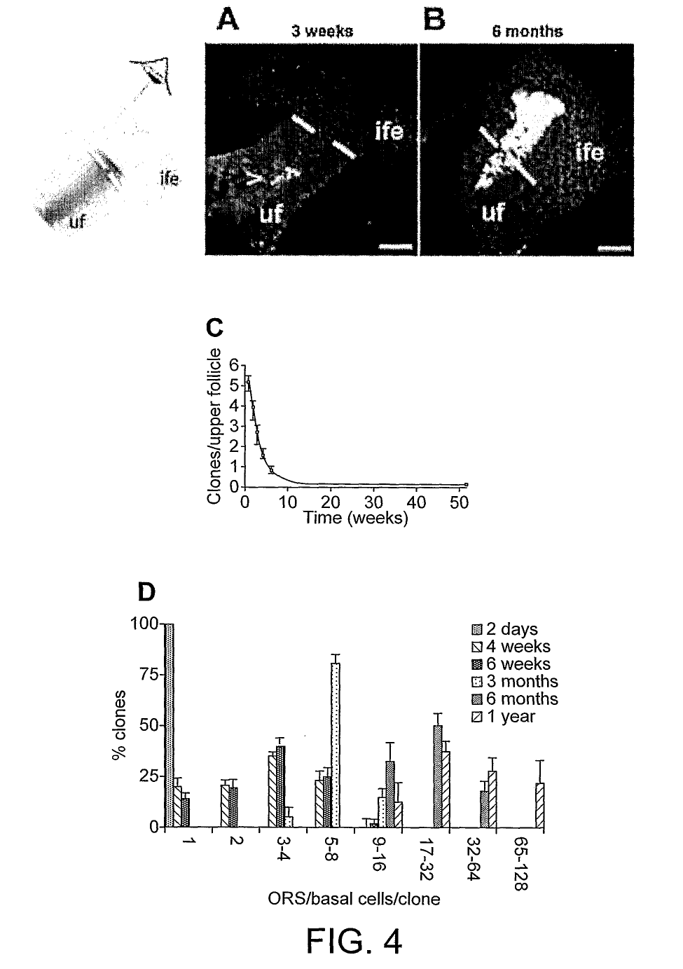

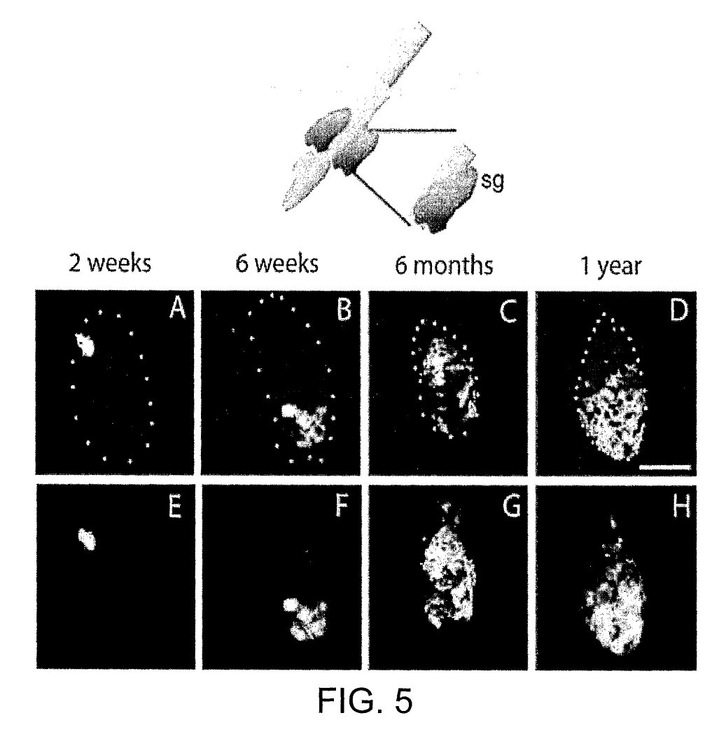

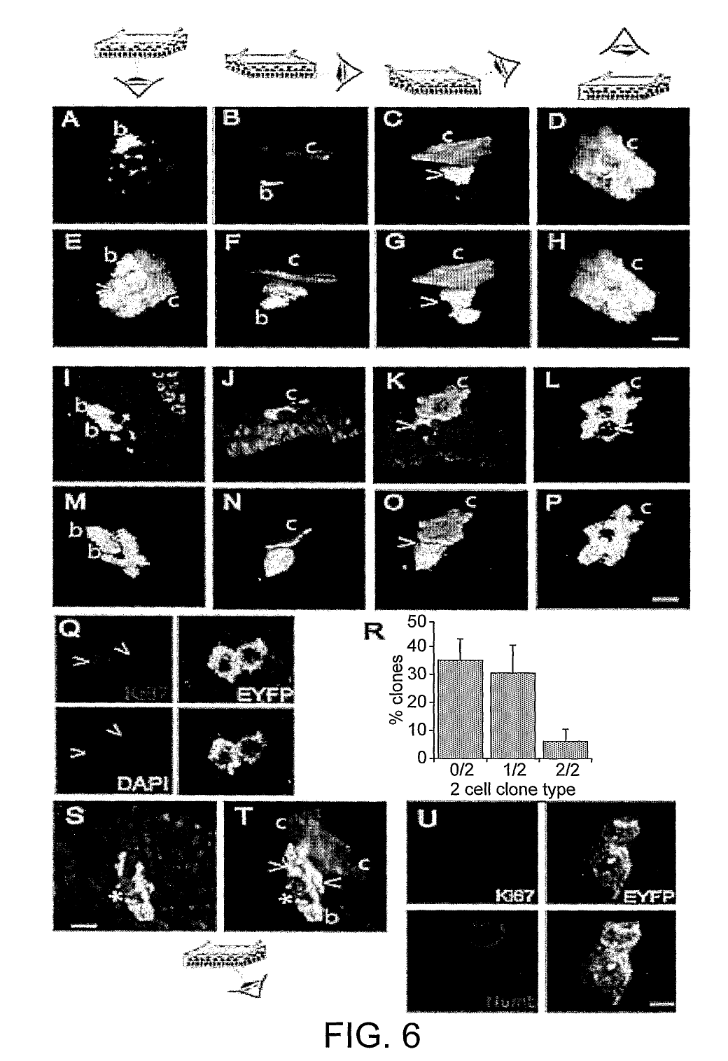

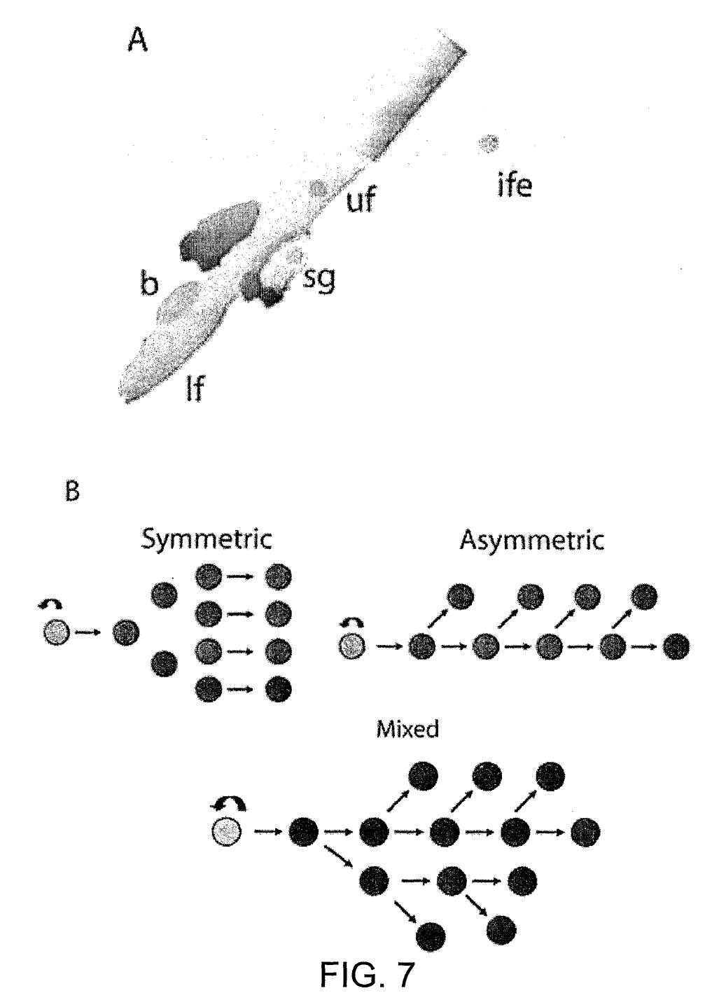

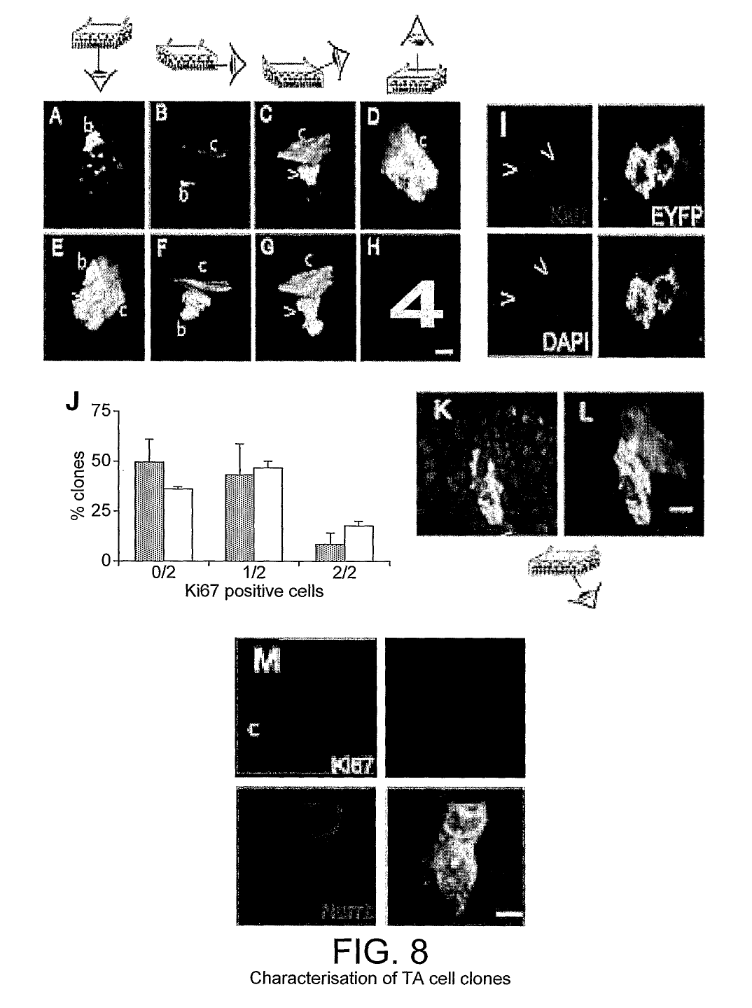

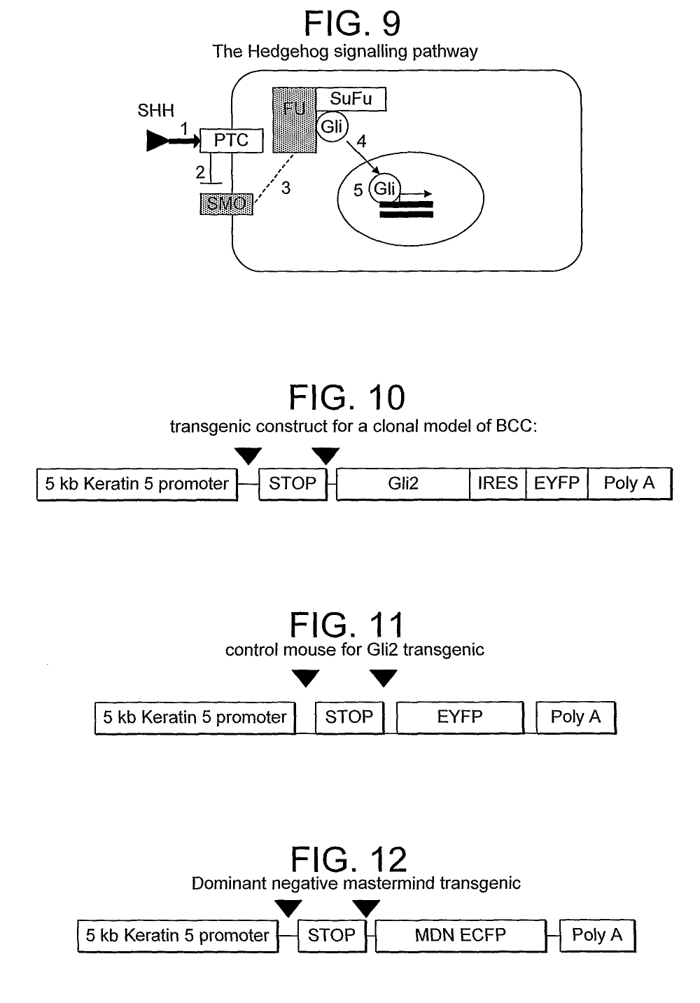

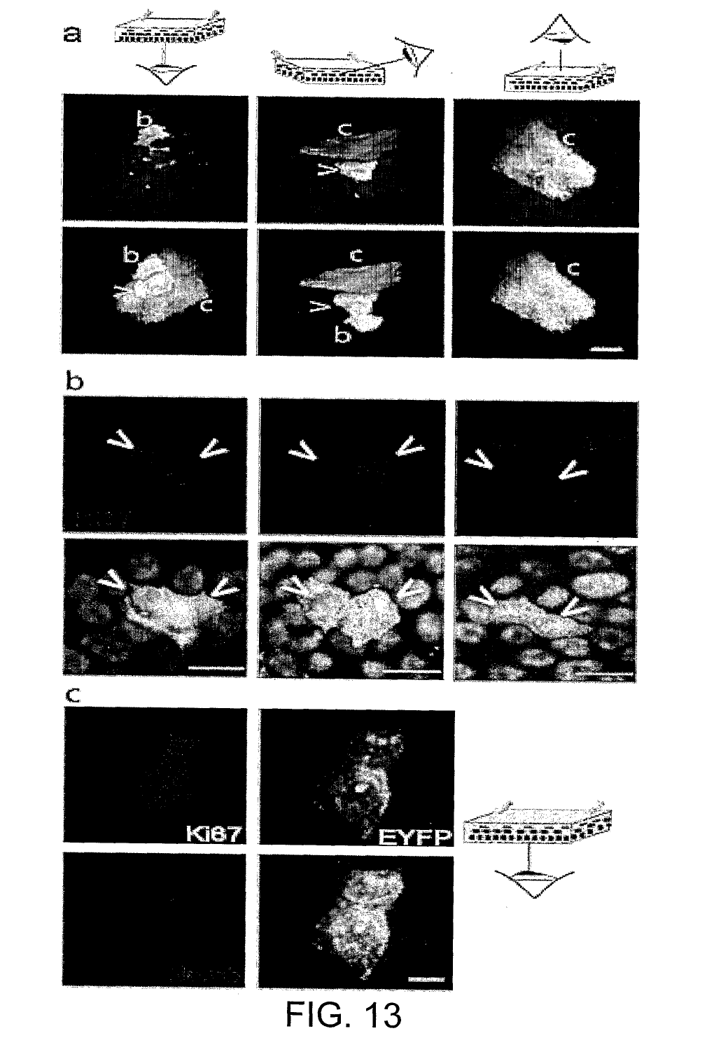

B: Change in the number of EYFP⁺ clones per unit area of interfollicular epidermis, defined as in FIG. 1D, over 1 year post induction. Error bars indicate standard error of the mean.

C: Size distribution of EYFP⁺ clones from 2 days post induction to 4 weeks. The total number of cells in each clone was counted at the time points shown. Error bars indicate the standard error of the mean.

D: Size distribution of EYFP⁺ clones from 2 days post induction to 1 year, expressed as the number of basal cells in each clone, at the time points shown. Error bars indicate the standard error of the mean.

FIG. 3 shows stem cell derived clones in interfollicular epidermis.

A,B: The epidermal proliferative unit (EPU) model of the IFE, which proposes that the epidermis is organised into hexagonal clonal units each of which supports the overlying stack of cornified and squamous cells (Mackenzie, 1970; Potten, 1976). A, view from external epidermal surface, showing a hexagonal squamous cell with the positions of the underlying basal cells. Blue indicates a stem cell, green the TA cells derived from it, and red a post mitotic basal cell about to leave the basal layer. The peripheral basal cells (denoted *) are more likely to be in cycle than the central cells within each unit. B, lateral view of the EPU, showing the central stem cell which maintains the column of overlying differentiated cells, basal cells are coloured as in A.

C: Projected. Z stack images of a typical EYFP⁺ clone 6 months post induction, cartoons indicate viewing angle, nuclei stained with DAPI (blue), EYFP is yellow. Scale bar represents 20 μm.

D: Projected Z stack image of basal surface of typical EYFP⁺ clone 6 months post induction stained for the β1 integrin subunit. Arrowheads indicate the position of basal cells expressing high levels of β1 integrin within the clone. Scale bar represents 20 μm.

E: Projected Z stack of basal layer of EYFP⁺ clone 6 months post induction, viewed from basal epidermal surface. Panels show: DAPI nuclear stain; blue; Ki67, red, yellow dotted outline indicates location of EYFP⁺ clone; EYFP⁺ clone, yellow, and the merged image. Scale bar represents 20 μm.

F: Distribution of EYFP⁺ clones in IFE over a 1 year time course following induction. The mean percentage of labelled interfollicular epidermal clones in regions 1 (solid squares) 2 (open circles) and 3 (solid triangles) is shown at each time point, error bars indicate SEM.

FIG. 4 shows proliferation of progenitors in the upper hair follicle

A,B: Projected Z stack images of epidermal whole mounts showing typical upper hair follicle clones from 3 weeks (A) and 6 months (B) post induction. The junction of upper hair follicle (uf) and IFE, viewed from exterior surface of epidermis is seen, as shown in the cartoon. Note the outer root sheath (ORS) of the upper hair follicle is continuous with the basal layer of IFE. Nuclei (blue) are stained with DAPI, EYFP is yellow, white dotted line indicates junction of IFE and hair follicle. Scale bar represents 20 μm.

C: Change in numbers of EYFP⁺ clones in the upper hair follicle over 1 year post induction, error bars show the standard error of the mean. D: Size of EYFP⁺ clones expressed as number of outer root sheath (ORS) and basal cells in each clone, for clones in the upper hair follicle. By 6 months typical clones have extended into the IFE. adjacent to the hair follicle and so contain both ORS and basal cells.

FIG. 5 shows sebaceous gland pregenitors

Projected Z stack images of sebaceous glands at the times shown post induction. EYFP⁺ cells appear yellow, nuclei abstained with DAPI, blue. Cartoon shows the angle of view. A-D: DAPI and EYFP; E-H, corresponding images with only EYFP channel shown. White dotted outline indicates the outer edge of the sebaceous gland. Scale bar represents 50 μm.

FIG. 6 shows characterisation of TA cell clones

A-D: Projected Z stacks of epidermal whole mounts, 3 weeks after recombination, cartoons indicate angle of view. The clones shown contain 3 cells (A-H) and 4 cells (I-P).

A-H: A three cell clone which contains one basal cell (b), and two suprabasal, differentiated cells, the uppermost of which has the appearance of a cornified layer cell (c), the position of the second suprabasal cell, which lies between the basal cell and the cornified cell is indicated by an arrowhead. A-D: EYFP, yellow and (DAPI) images. E-H: images from the same angle of view as in A-D, but with only the EYFP channel shown. A, E, view from basal surface; B, F, lateral view; C, G: oblique view; D, H view from external surface. Scale bar corresponds to 20 μm.

I-H: A four cell clone which contains two basal cells (b), and two suprabasal, differentiated cells, the uppermost of which has the appearance of a cornified layer cell (c), the position of the second suprabasal cell, which lies between the basal cell and the cornified cell is indicated by an arrowhead. I-L: EYFP, yellow and (DAPI) images. M-P: images from the same angle of view as in I-L, but with only the EYFP channel shown. I, M, view from basal surface; L. N, lateral view; K, O: oblique view; L, P view from external surface. Scale bar corresponds to 20 μm.

Q: Projected Z stack image of 2 cell clone, containing 2 basal cells, 3 weeks after recombination, viewed from the basal epidermal surface, stained for the proliferation marker: Panels show: Ki67 staining, red, arrowheads indicate position of EYFP⁺ cells; nuclear stain DAPI (blue), arrowheads indicate position of EYFP⁺ cells; EYFP, yellow, and merged image. Scale bar represents 5 μm.

R: Proportion of 2 cell clones showing symmetric and asymmetric proliferation. The percentages of 2 clones expressing the Ki67 proliferation marker none, one or both cells, 2 weeks after induction, is shown. Error bars show the standard deviation.

S,T: Projected Z stack image of a 6 cell EYFP⁺ clone 6 weeks post induction, stained for the proliferation marker cdc6 (red), viewed obliquely from the basal epidermal surface, as shown in cartoon. The clone contains 2 basal cells, one of which is cdc6 positive (*) and one negative (b), 2 suprabasal cells (arrowheads) and 2 cornified cells (c). S: image showing cdc6 with EYFP, yellow and (DAPI) channels; T, image showing only cdc6 and EYFP channels, Scale bar corresponds to 20 μm.

U: Confocal image of 2 cell clone, containing 2 basal cells, 3 weeks after recombination, viewed from the basal epidermal surface, stained for the proliferation marker Ki67 (blue) and numb (red), EYFP is yellow. Scale bar represents 5 μm.

FIG. 7 shows stem cell and TA cell fate in the epidermis.

A: Model showing the stem cell populations that support the normal epidermis. Location of functioning stem cells in the adult epidermis. Stem cells (blue), reside in the sebaceous glands (sg), upper hair follicle (uf) and interfollicular epidermis (IFE) and the support clonal units (all shown yellow) as shown. These stem cells are independent of those in the bulge, b, which maintains the lower hair follicle (lf).

B: Models of TA cell fate. Stem cells, (blue) divide to generate TA cells and stem cells. TA cells (green) proliferate for a limited number of divisions after which all their progeny differentiate to generate postmitotic cells (red). 3 types of TA cell behaviour are illustrated; symmetrical proliferation and differentiation, asymmetrical behavious in which each TA cell division generates a post mitotic and a proliferating cell, until a final division when both cells differentiate, and a mixed pattern. Whilst other precursors, such as O2A cells, exhibit symmetric proliferation, followed by synchronous differentiation, epidermal TA cells exhibit mixed symmetric and asymmetric behaviour.

FIG. 8 shows photomicrographs of cells. See description for FIG. 6 for further detail.

FIG. 9 shows a diagram of the hedgehog signalling pathway

FIG. 10 shows a diagram of a genetic construct.

FIG. 11 shows a diagram of a genetic construct.

FIG. 12 shows a diagram of a genetic construct.

FIG. 13 shows photomicrographs of symmetric cell cycle exit in epidermal progenitors. 13a: Projected Z stack images of a three cell clone containing one basal cell (b), and two suprabasal cells (a cornified layer cell (c), and a second suprabasal cell indicated by the arrowhead). Cartoon shows the angle of view. Upper panels: EYFP, yellow and DAPI, blue; lower panels are corresponding images with only EYFP shown. Scale bar 20 μm.

13b: Clones consisting of 2 basal cells, 3 weeks after recombination, viewed from the basal epidermal surface; stained for the proliferation marker Ki67 (red), DAPI (blue), and EYFP (yellow), arrowheads indicate position of EYFP labelled cells. Three types of clone are shown, with two, one and zero Ki67 positive cells. Scale bar 10 um.

13c: 2 cell clone, containing 2 nasal cells, 3 weeks after recombination, viewed from the basal epidermal surface, stained for the proliferation marker Ki67 (blue), numb (red) and EYFP yellow. Scale bar 5 um.

The examples make use of the following general techniques:

### Animals and Sample Preparation

The generation of Ahcre^{ERT} and R26^{EYFP/EYFP} mice has been described previously (Kemp et al (2004) Nucleic Acids Res 32, e92; Srinivas et al (2001) BMC Dev Biol 1, 4). To induce ere expression AhcreERT R26^{EYFP/wt} heterozygote animals were given a single intraperitoneal injection of 80 mg/kg β-napthoflavone (SIGMA) and 1 mg tamoxifen free base (MP Biomedicals) dissolved in corn oil. To prepare epidermal wholemounts, mice were killed, and then tail skin was cut into 0.5 cm sections and incubated in 5 mM EDTA/PBS for 4 h at 37 C. Epidermis was then peeled of the dermis and fixed in 4% paraformaldehyde for 1 h at room temperature. Fixed epidermal sheets were stored in PBS at 4C.

### Immunostaining and Imaging

Epidermal sheets were blocked and permeabilized by incubation in PB buffer containing 0.5% BSA 0.25% Fish skin gelatin, 0.5% Triton X-100 and Goat/Donkey/Rat serum as appropriate in PBS for 1 h. Primary antibodies were diluted in PB buffer and epidermal sections incubated overnight at room temperature on rocking platform then washed 4×1 h in PBS/0.2% Tween 20. Secondary antibodies were diluted 1:250 in PB buffer, sections incubated overnight, then washed 4×1 h. Samples were rinsed in distilled water and mounted. Staining of wholemounts with mouse monoclonal antibodies was performed using the M.O.M kit (Vector Laboratories) according to the manufacturer's instructions except that staining with primary antibody was extended to 3 h and staining with secondary antibody to 1 h. The following primary antibodies were used GFP Rabbit polyclonal (Abcam), anti GFP conjugated to Alexa 488 or 555 (Molecular Probes), anti Ki67 Rabbit polyclonal (Abcam), mouse monoclonal anti cdc6 (Molecular Probes), CD29 anti β1-integrin Rat monoclonal 9EG7 (BD-Pharmingen), anti numb (Abcam) 1:200. Secondary antibodies were from Molecular Probes.

Images were acquired using a Zeiss 510 confocal microscope. Scans are presented as Z-stack projections where 30-120 optical sections in 0.2-2 μm increments were captured.

### EXAMPLE 1

### Imaging a Clonal Cell Line in a Test Animal

Mice transgenic for an inducible form of cre recombinase (Ahcre^{ERT}) were crossed onto the R26^{EYFP/EYFP} reporter strain in which a conditional allele of Enhanced Yellow Fluorescent Protein (EYFP) has been targeted to Rosa26 locus by homologous recombination (FIG. 1B, Kemp et al (2004) Nucleic Acids Res 32, e92; Srinivas et al (2001) BMC Dev Biol 1, 4). In the resultant Ahcre^{ERT} R26^{EYFP/wt} heterozygote animals, EYFP is expressed in the epidermal cells following a single injection of βNF and tamoxifen at 6-9 weeks of age. At intervals after induction, mice were sacrificed for analysis. Cells expressing EYFP (EYFP⁺) and their EYFP⁺ progeny were detected by confocal microscopy and reconstruction of wholemount epidermis, in which the entire epidermis is detached from the underlying dermis, allowing all cells in the tissue to be imaged at single cell resolution (Braun et al (2003) Development 130, 5241-5255). In this study we used tail skin epidermis from the proximal 2 cm of the tail. The patterned organisation of tail epidermis, with regularly spaced clusters of hair follicles separated by IFE enables quantitative analysis of EYFP⁺ cells; here we define a unit area of IFE as shown (FIG. 1 C-F). 1 unit area measures 282,000+/−2300 μm2 and contains 4870+/−400 (mean +/− SD) basal layer cells.

When Ahcre^{ERT} R26^{EYFP/wt} mice are treated with multiple doses of βNF-and tamoxifen, a high level of recombination is seen in the upper hair follicle, sebaceous glands and keratinocytes in all layers of the interfolliclular epidermis. However, titration of drug doses produced a proportionately lower frequency of recombination; by treating with a single dose of both inducing drugs, EYFP expression was induced in 1 in 635 basal cells in the IFE, 1 in 27 outer root sheath cells-in the upper hair follicle and 1 in 35 cells in the sebaceous glands at 1 week post induction. Crucially, there was no background recombination prior to drug treatment and no labelled cells were detected in the bulge region or in the lower hair follicle, consistent with the lack of activity of the Ah promoter in these areas (FIG. 1C, 2A).

The behaviour of EYFP⁺ cells and the resultant clones in IFE was examined over 1 year following induction. Proliferation is confined to cells in the basal layer of the epidermis, so we began by examining the proliferation of EYFP⁺ basal cells. The appearance of typical clones in wholemount preparations, viewed from the basal surface, is shown in FIG. 2A. At 2 days post induction, only single EYFP⁺ cells were seen in all layers of the IFE; the labelled cells were widely separated. Subsequently, EYFP⁺ basal cells proliferated to give EYFP+ clones that remained cohesive and expanded progressively in size (FIG. 2A).

As the basal epidermis contains both TA cells and stem cells, two types of behaviour of would be predicted for EYFP⁺ basal cell clones. The majority of clones, derived from TA cells, would be expected to be of small size and be lost as all cells in the clone underwent terminal differentiation, whilst a small number of stem cell derived clones would persist in the tissue for an extended period. The number of EYFP⁺ clones in the basal layer of a unit area of IFE falls substantially over the weeks following induction, to less than 50% of the peak value by 4 weeks and to only 3.8% by 3 months (FIG. 2B). This is consistent with 96% of the EYFP⁺ basal cells at baseline being either TA cells or post mitotic, differentiated basal cells. Strikingly, the number of labelled clones remains almost constant between 3 months and 1 year (3.8% at 3 months, 3.2% at 1 year. This indicates these long lived EYFP⁺ clones originate from labelled stem cells, which are able maintain the clones for at least half the lifetime of the animal.

The time taken, for TA cell clones and post mitotic basal cells to be shed from the epidermis indicates that the epidermal transit time, i.e. the time taken for TA cell to complete proliferation, differentiate, migrate through the suprabasal epidermis to the epidermal surface, is 6-12 weeks (FIG. 2C). This is substantially longer than previous estimates which have been in the range of 5-9 days.

Previous estimates of cell proliferation in vivo have been based on tritiated thymidine labelling using methods such as the first wave of labelled mitoses technique. However the interpretation these methods has proven controversial. Using, wholemount imaging, the proliferation of cohorts of EYFP⁺ cells can be visualised directly. As single cells are labelled at the start of the experiment, the total number of cells, both basal and superbasal, in each EYFP⁺ clone gives a measure of its proliferation. Data from the first 4 weeks following induction, when over 90% of proliferating EYFP⁺ clones are derived from TA cells are shown in FIG. 2C.

There are four striking features of the how clone size varies with time. i) The rate of expansion of different clones varies substantially. At 2 weeks, proliferating EYFP⁺ clones ranged from 2 to 8 cells in size whilst at 4 weeks the range was from 2 to 18 cells (FIG. 2C). There is no change in the proportion of clones containing between 2 and 6 cells between 2 and 4 weeks, and by 6 weeks all 2, 3 and 4 cell clones are negative for the proliferation markers Ki67 and cdc6. This is consistent with these clones being derived from TA cells, whose progeny all undergo terminal differentiation after only a few rounds of cell division, ii) The rate of clone expansion is significantly slower than would be predicted from tritiated thymidine studies, which estimate the average cell cycle time in mouse back skin epidermis as 100-120 hours.

Whilst many clones cease proliferating, the size of the largest 10% of EYFP clones increases from a range of 6-8 cells at 2 weeks to 9-18 cells at 4 weeks, iii) TA cell proliferation in vivo is also dramatically slower than that seen with primary cell cultures; TA cell clones show a similar size distribution to that seen in vivo at 4 weeks after only 3 days in culture, iv) FIG. 2C demonstrates that many clones contain odd numbers of cells. The distribution of clone sizes indicates that clones do net expand geometrically in powers of 2, (1,2 4, 8, 16 etc), but rather increase in size in an arithmetic progression, (1,2,3,4,5 etc), suggesting that some TA cell divisions generate one differentiated cell and one cell that continues to proliferate; this is discussed further below.

At time points later than 4 weeks post induction EYFP⁺ clones accumulate anucleate cornified cells, making it impossible to score cell numbers accurately (cf FIG. 3C); We therefore counted the number of basal cells, in each clone at later time points. Clone numbers fall only slightly from 3 months to 1 year, indicating the majority of clones at these time-points are derived from stem cells (FIG. 2B, D). Again, a wide range of clone sizes is seen. This may reflect the different proliferative potential of long lived clones, such as is seen human primary keratinocyte cultures grown at clonal density. A continuum of clone sizes and appearances is seen in these cultures; when subcloned, some colonies have very high proliferative potential (holoclones) whereas others exhibit TA cell type behaviour (paraclones), whilst the remainder exhibit intermediate proliferative potential (meroclones). The rate of clone expansion falls dramatically, with the largest 10% clones increasing in size from a range of 45-55 cells at 3 months to 120-160 cells at 1 year. The observation that maximum clone size continues to increase between 6 and 12 months, is consistent with labelled clones expanding to occupy space vacated by the loss and/or decrease in size of adjacent clonal units supported by unlabelled stem cells. This parallels the age related loss of proliferative potential seen in cultures of human keratinocytes, where cultures of epidermis from donors aged over 60 lack the large, self renewing “holoclone” type colonies which characterise cultures of neonatal skin.

We went on to examine the structure of long lived stem cell derived clonal units. Early models of epidermal organisation proposed that the epidermis consists of epidermal proliferative units (EPU) in which a single central stem cell supports 9 surrounding basal cells and the overlying column of suprabasal cells (FIG. 3A, B). Proliferating TA cells lie around the margins of each EPU. We examined long lived clonal units for features of an EPU. EYFP⁺ clones at 6 months and 1 year post induction are larger than predicted by the EPU model, containing up to 150 basal cells (FIG. 2D, 3A). Markers for stem cells in IFE are limited, but in human IFE stem cells represent a subpopulation of the keratinocytes that express high levels of β1 integrin. In mouse tail whole mounts we found 16%+/−3.5% (mean+/−standard deviation) of cells were β1 integrin bright, whilst 3% of basal cells are stem cells. The EPU model predicts that a centrally located cell expressing high levels of β1 integrin. would be found within each clone. We found multiple β1 integrin bright cells in each clonal unit at 6 months and 1 year post induction, but these have a highly a variable distribution in clones (FIG. 3D). This observation is in keeping with the lack of any pattern in the distribution of label retaining cells in tail skin epidermis. Finally, staining with the proliferation marker Ki67 does not reveal evidence of any pattern of cellular proliferation within labelled clonal units at 6 and 12 months post induction (FIG. 3E). These results do not support the existence of EPU in tail epidermis.

Next we addressed the issue of whether the IFE is maintained by stem cells in the bulge, or the upper hair follicle, the IFE itself, or by all three of these sites. The regular pattern of hair follicles in mouse tail skin enabled us to examine whether the distribution of clones across the IFE varied with time. Each unit area of IFE was divided into three equal areas and the proportion of labelled clones in each area scored at different time points (FIG. 1D-F, 3F). No bulge cells were labelled with EYFP, so if the IFE is maintained by the bulge stem cells a progressive loss of labelled IFE clones would be seen over the year of the experiment, as they were replaced by unlabelled clones from the bulge. However, we found that the percentage of labelled clones in each area did not change from baseline, even in the epidermis lying furthest from hair follicles, indicating IFE was maintained by stem cells independent of the bulge over the year of the experiment.

The observation that adult IFE contains stem cells which are independent from hair follicle stem cells does not exclude a role for hair follicle stem cells in maintaining the epidermis immediately adjacent to the hair follicle, where the outer root sheath of the follicle is in continuity with the basal layer of the IFE. Double labelling studies have shown that proliferating cells migrating from the upper hair follicle into the adjacent IFE in neonatal mice. We therefore examined EYFP⁺ clones in the upper follicle (FIG. 4). The number of EYFP+ clones in the upper hair follicle fell in a similar manner to that seen in IFE (FIG. 4C). 97% of EYFP⁺ clones behave like TA cell clones, being lost through terminal differentiation by 12 weeks post induction (FIG. 4C). Clone numbers remain constant between 6 and 12 months, however, indicating the remaining 3% of clones were derived from stem cells in the upper follicle. Likewise the size of upper follicle clones expands in a manner similar to those in the IFE (FIG. 4 A, B, D). Strikingly labelled clones extending from the upper follicle into the adjacent IFE are seen by 6 months post induction, and these persist until at least the 12 month time point (FIG. 4B). Thus the epidermis adjoining the hair follicle is maintained by upper follicle stem cells, which are independent of the bulge during a year of adult life.

We also examined progenitors in sebaceous glands. 94% of sebaceous glands contained one or more EYFP⁺ cells at 2 days after induction; by 1 year the percentage of labelled glands had fallen to 2.3%. EYFP⁺ clones were seen that progressively expanded during the course of the experiment, so that by 1 year typical glands contained over 90% EYFP⁺ cells (FIG. 5 A-H). Again, this indicates the presence of stem cells independent of bulge stem cells in sebaceous glands, consistent with previous observations in retroviral marking studies.

Finally we investigated the structure of TA cell clones. There are no molecular markers for TA cells, but the analysis of clone numbers performed above indicates that over 90% of clones containing 2 or more cells at the 2-4 week post induction time are shed from the epidermis by 12 weeks post induction, indicating that they derive from TA cells. TA cells in other systems, such as O2A oligodendrocyte precursors, undergo several rounds of synchronous proliferation, after which all cells undergo simultaneous terminal differentiation; this process is symmetrical in that the fate of both daughter cells after each cell division is identical. Epidermal TA cells have been thought to behave in the same manner; with 3 rounds of TA cell division generating 8 post mitotic keratinocytes within each EPU. Typical examples of a TA cell clones, containing 3 and 4 cells, 3 weeks post induction are shown in FIG. 6A-P;. FIG. 6 A-H shows a 3 cell clone, containing one basal cell and two suprabasal, differentiated cells, one of which has the flattened appearance of a cornified layer cell. The existence of such a clone indicates that unlike oligodendrocyte precursors, epidermal TA cells differentiate in an asynchronous manner. The 4 clone illustrated also exhibits asynchronous differentiation (FIG. 6I-P). This clone contains 2 basal cells (FIG. 6 I, M), a suprabasal cell (FIG. 6 J,K) and a flattened cornified layer cell FIG. 6 L,P). The 2 cell divisions that have occurred since induction, have thus generated two terminally differentiated, suprabasal cells and 2 cells that remain in the basal layer, indicating asymmetric cell division, in which daughter cells have different fates.

Asymmetry in TA cell fate was also apparent when epidermal TA cell proliferatron was examined. In 2 cell clones examined 3 weeks after induction, 30.5% +/−6.5% (mean+/−SEM) had one cycling and one non cycling cell as assessed by Ki67 staining, whilst in the remaining clones both cells were either Ki67 negative, or Ki67 positive (FIG. 6 Q,R). Staining of larger clones reveals cycling basal cells, expressing the cell cycle markers cdc6 or Ki67, in clones that also contain terminally differentiated cells (FIG. 6 S,T). Taken together these results indicate that TA cells undergo, both symmetric and asymmetric cell division. Symmetric divisions result in 2 cycling daughter cells whilst asymmetric divisions generate one proliferating and one daughter committed to terminal differentiation (FIG. 2R). The proportion of asymmetric cell division seen is consistent with the observation that many clones contain odd numbers of cells 2-4 weeks post induction (FIG. 2C).

Asymmetric cell division in murine CNS and muscle progenitors is regulated by numb protein, which segregates to one of the two dividing cells, blocking Notch signalling. Immunostaining of 2 cell clones revealed numb protein localised to one of the two cells in proliferating, Ki67 positive cell clones, consistent with asymmetric TA cell division being regulated by numb (FIG. 6 U).

TA cells have been hypothesised to undergo 3-5 rounds of cell division followed by synchronous differentiation. To test this prediction we examined the appearance of clones at 3 weeks, over 90% of which are lost by 12 weeks post induction. Significantly, clones comprising three or more cells contained both basal and suprabasal cells, indicative of asynchronous terminal differentiation (FIG. 13a). Furthermore, the immunostaining of clones consisting of two basal cells reveals that a single cell division may generate either one cycling and one non-cycling daughter, or two cycling daughters, or two non-cycling daughters (FIG. 13b). This raises the question of whether there is asymmetric cell division within the basal plane. Three-dimensional imaging of wholemount epidermis revealed that only 3% of mitotic spindles lie perpendicular to the basal layer indicating that, in contrast to embryonic epidermis, the vast majority of epidermal progenitor cell (EPC) divisions generate two basal layer cells. Such asymmetric cell divisions within a plane of cells have been observed in the Drosophila peripheral nervous system and Zebra Fish retinal precursors. The observation of asymmetric partitioning of numb protein, which marks asymmetric division in neural and myogenic precursors, in clones containing two basal cells leads us to conclude that planar-orientated asymmetric division also occurs in the epidermis (FIG. 13c). EPC behaviour thus differs substantially from that predicted for TA cells and observed in committed precursors in other systems.

Inducible genetic marking complements previous stem cell studies using label retention, which identify quiescent stem cells but give no information on the clonal units maintained by active stem cells. The observations presented here suggest that the IFE, hair follicle and sebaceous glands contain stem cells, which are independent of bulge stem cells over a one year period (FIG. 7). A second surprising observation is that, in contrast to other progenitor cells, such as O2A oligodendrocyte precursors, which exhibit symmetric proliferation and differentiation, epidermal TA cells exhibit both symmetric and asymmetric cell division (FIG. 7B). This gives rise to a pattern of clone expansion that generates odd numbers of cells in numerous TA derived clones, and explains the slow rate of TA clone expansion (FIG. 2C).

The invention can be further applied to quantitative analysis of stem and TA cell proliferation and fate in other tissues in which molecular markers of stem and TA cells are not available. This system also offers a way to determine the effect of conditional expression of growth regulatory and oncogenic genes in individual adult epidermal progenitor cells in a wild type background.

### EXAMPLE 2

### Inducible Clonal Targeting in Adult Mouse Epidermis

We show a system that allows the controlled induction of specific mutations in individual epidermal cells in a wild type background, and enables the mutated clones and their progeny to be followed over an extended period. To do this we exploited a transgenic mouse line (Ahcre^{ERT}) which expresses a tamoxifen regulated ere reeombinase-mutant oestrogen receptor fusion protein under the control of the CYPA1A promoter (Kemp et al. 2004). CYPA1A is normally tightly repressed, out is induced following administration of the non genetoxic xenobiatic B-napthoflavone (B-NF) in several tissues including the epidermis and the squamous oesophagus. Dual transcriptional and post-translational control of cre expression results in no detectable background recombination in adult epidermis in the absence of treatment with the inducing drugs. High doses of the inducing drugs result in widespread recombination in the upper hair follicle and interfollicular epidermis, but by titrating down the doses of B-NF and tamoxifen it is possible to induce low frequency recombination at these sites; for example, recombination occurs in 1 in 635 cell's in interfollicular epidermis. We have characterised the fate of the targeted cells.

### EYFP Clonal Marking in Adult Mouse Epidermis

Ahcre^{ERT} mice were crossed onto the R26^{EYFP/EYFP} reporter strain in which a conditional allele of Enhanced Yellow Fluorescent Protein (EYFP) has been targeted to Rosa26 locus by homologous recombination (see Figures; Kemp et al 2004). In the resultant Ahcre^{ERT} R26^{EYFP/wt} heterozygote animals, EYFP is expressed in the epidermal cells following a single injection of B-NF and tamoxifen at 6-9 weeks of age. At intervals after induction, mice were sacrificed for analysis. Cells expressing EYFP (EYFP⁺) and their EYFP⁺ progeny were detected by confocal microscopy and reconstruction of wholemount epidermis, in which the entire epidermis is detached from the underlying dermis, allowing all cells in the tissue to be imaged at angle, cell resolution (see Figures; Braun et al. 2003). We have analysed tail skin epidermis from the proximal 2 cm of the tail. The patterned organisation of rail epidermis, with regularly spaced clusters of hair follicles separated by IFE enables quantitative analysis of EYFP⁺ cells; we defined a unit area of IFE as shown (see Figures). 1 unit area measures 282,000+/−2300 um2 and contains 4870+/−400 (mean+/−SD) basal layer cells.

Crucially, there was no background recombination prior to drug treatment and no labelled cells were detected in the bulge region or in the lower hair follicle, consistent, with the lack of activity of the Ah promoter in these areas. Using doses of B-NF and tamoxifen as given under ‘Animals and Sample Preparation’ above, 1 in 635 cells are targeted in the tail whilst 1 in 38 cells are targeted in mouse back skin IFE.

### Fate of Labelled Cells

The behaviour of EYFP⁺ cells and the resultant clones in IFE was examined over 1 year following induction. Proliferation is confined to cells in the basal layer of the epidermis, so we began by examining the proliferation of EYFP⁺ basal cells. The appearance of typical clones in wholemount preparations of tail IFE, viewed from the basal surface, is shown in the Figures. At 2 days post induction, only single EYFP⁺ cells were seen in all layers of the IFE; the labelled cells were widely separated. Subsequently, EYFP⁺ basal cells proliferated to give EYFP+ clones that remained cohesive and expanded progressively in size (FIG. 2A).

As the basal epidermis contains both TA cells and stem cells, two types of behaviour would be predicted for EYFP⁺ basal cell clones. The majority of clones, derived from TA cells, would be expected to be of small size and be lost as all cells in the clone underwent terminal differentiation, whilst a small number of stem cell derived clones would persist in the tissue for an extended period. The number of EYFP⁺ clones in the basal layer of a unit area of IFE falls substantially over the weeks following induction, to less than 50% of the peak value by 4 weeks and to only 3.8% by 3 months (FIG. 2B). This is consistent with 96% of the EYFP⁺ basal cells at baseline being either TA cells or post mitotic, differentiated basal cells. Strikingly, the number of labelled clones remains almost constant between 3 months and 1 year (3.8% at 3 months, 3-2% at 1 year. This indicates these long lived EYFP⁺ clones originate from labelled stem cells, which are able maintain the clones for at least half the lifetime of the animal. Analysis of back skin IFE by frozen sections reveals a similar decay in clone numbers, with 6% of labelled clones persisting for one year.

There are four striking features of how clone size varies with time, i) The rate of expansion of different clones varies substantially. At 2 weeks, proliferating EYFP⁺ clones ranged from 2 to 8 cells in size whilst at 4 weeks the range was from 2 to 18 cells (FIG. 2C). There is no change in the proportion of clones containing between 2 and 6 cells between 2 and 4 weeks, and by 6 weeks all 2, 3 and 4 cell clones are negative for the proliferation markers Ki67 and cdc6. This is consistent with these clones being derived from TA cells, whose progeny all undergo terminal differentiation after only a few rounds of cell division. ii) The rate of clone expansion is significantly slower than would be predicted from tritiated thymidine studies, which estimate the average cell cycle time in mouse back skin epidermis as 100-120 hours. Whilst many clones cease proliferating, the size of the largest 10% of EYFP⁺ clones increases from a range of 6-8 cells at 2 weeks to 9-18 cells at 4 weeks, iii) TA cell proliferation in vivo is also dramatically slower than that seen with primary cell cultures; TA cell clones show a similar size distribution to that seen in vivo at 4 weeks after only 3 days in culture. iv) FIG. 2C demonstrates that many clones contain odd numbers of cells. The distribution of clone sizes indicates that clones do not expand geometrically in powers of 2, (1,2 4, 8, 16 etc), but rather increase in size in an arithmetic progression, (1,2,3,4,5 etc), suggesting that some TA cell divisions generate one differentiated cell and one cell that continues to proliferate; this is discussed further below. This result is consistent with the recent observation that keratinocytes divide asymmetrically in embryonic epidermis.

### Short Lived, Transit Amplifying Clones

We then investigated the structure of TA cell clones. There are no molecular markers for TA cells, but the analysis of clone numbers performed above indicates that over 90% of clones containing 2 or more cells at the 2-4 week post induction time are shed from the epidermis by 12 weeks post induction, indicating-that they derive from TA cells. TA cells in other systems, such as O2A oligodendrocyte precursors, undergo several rounds of synchronous proliferation, after which all cells undergo simultaneous terminal differentiation; this process is symmetrical in that the fate of both daughter cells after each cell division, is identical. Epidermal TA cells have been thought to behave in the same manner, with 3 rounds of TA cell division generating 8 post mitotic keratinocytes within each EPU, A typical examples of a TA cell clones, containing 3 cells, 3 weeks post induction are shown in FIG. 8 A-H, where a 3 cell clone, containing one basal cell and two suprabasal, differentiated cells, one of which has the flattened appearance of a cornified layer cell is shown. The existence of such a clone indicates that unlike oligodendrocyte precursors, epidermal TA. cells differentiate in an asynchronous manner.

Asymmetry in TA cell fate was also apparent when epidermal TA cell proliferation was examined. In 2 cell clones examined 3 weeks after induction, 30.5% +/−6.5% (mean+/−SBM) had one cycling and one non cycling cell as assessed by Ki67 staining, whilst in the remaining clones both cells were either Ki67 negative or Ki67 positive. Staining of larger clones reveals cycling basal cells, expressing the cell cycle markers cdc6 or Ki67, in clones that also contain terminally differentiated cells. Taken together these results indicate that TA cells undergo both symmetric and asymmetric cell division. Symmetric divisions result in 2 cycling daughter cells whilst asymmetric divisions generate one proliferating and one daughter committed to terminal differentiation. The proportion of asymmetric cell division seen is consistent with the observation that many clones contain odd numbers of cells 2-4 weeks post induction (FIG. 2C).

Asymmetric cell division in murine CNS and muscle progenitors is regulated by numb protein, which segregates to one of the two dividing cells, blocking Notch signalling. Immunostaining of 2 cell clones revealed numb protein localised to one of the two ceils in proliferating, Ki6-7 positive cell clones, consistent with asymmetric TA cell division being regulated by numb.

### Long Lived, Stem Cell Derived Clones

At time points later than 4 weeks post induction EYFP⁺ clones accumulate anucleate cornified cells, making it impossible to score cell numbers accurately; we therefore counted the number of basal cells in each clone at later time points. Clone numbers fall only slightly from 3 months to 1 year, indicating the majority of clones at these time points are derived from stem cells (FIG. 2B, D). Again, a wide range of clone sizes is seen. This may reflect the different proliferative potential of long lived clones, such as is seen human primary keratinocyte cultures grown at clonal density. A continuum of clone sizes and appearances is seen in these cultures; when subcloned, some colonies have very high proliferative potential (holoclones) whereas others exhibit TA cell type behaviour (paraclones), whilst the remainder exhibit intermediate proliferative potential (meroclones). The rate of clone expansion falls dramatically, with the largest 10% of clones increasing in size from a range of 45-55 cells at 3 months to 120-160 cells at 1 year. The observation that maximum clone size continues to increase between 6 and 12 months, is consistent with labelled clones expanding to occupy space vacated by the loss and/or decrease in size of adjacent clonal units supported by unlabelled stem cells. This parallels the age related loss of proliferative potential seen in cultures of human keratinocytes, where cultures of epidermis from donors aged over 60 lack the large, self renewing “holoclone” type colonies which characterise cultures of neonatal skin.

We went on to examine the structure of long lived stem cell derived clonal units. Early models of epidermal organisation proposed that the epidermis consists of epidermal proliferative units (EPU) in which a single central stem cell supports 9 surrounding basal cells and the overlying column of suprabasal cells (FIG. 8 A, B). Proliferating TA cells lie around the margins of each EPU. We examined long lived clonal units for features of an EPU. EYFP⁺ clones at 6 months and 1 year post induction are larger than predicted by the EPU model, containing up to 150 basal cells (FIG. 2D, 8A). Markers for stem cells in IFE are limited, but in human IFE stem cells represent a subpopulation of the keratinocytes that express high levels of B1 integrin. In mouse tail whole mounts we found 16%+/−3.5% (mean+/−standard deviation) of cells were B1 integrin bright, whilst 3% of basal cells are stem cells. The EPU model predicts that a centrally located cell expressing high levels of B1 integrin would be found within each clone. We found multiple B1 integrin bright cells in each clonal unit at 6 months and 1 year post induction, but these have a highly a variable distribution in clones. This observation is in keeping with the lack of any pattern in the distribution of label retaining cells in tail skin epidermis. Finally, staining with the proliferation marker Ki67 does not reveal evidence of any pattern of cellular proliferation within labelled clone units at 6 and 12 months post induction (FIG. 8E). These results do not support the existence of EPU in tail epidermis.

Next we addressed the issue of whether the IFE is maintained by stem cells in the bulge, or the upper hair follicle, the IFE itself, or by all three of these sites. The regular pattern of hair follicles in mouse tail skin enabled us to examine whether the distribution of clones across the IFE varied with time. Each unit area of IFE was divided into three equal areas and the proportion of labelled clones in each area scored at different time points (FIG. 8F). No bulge cells were labelled with EYFP, so if the IFE is maintained by the bulge stem cells a progressive loss of labelled IFE clones would be seen over the year of the experiment, as they were replaced by unlabelled clones from the bulge. However, we found that the percentage of labelled clones in each area did not change from baseline, even in the epidermis lying furthest from hair follicles, indicating IFE was maintained by stem cells independent of the bulge over the year of the experiment. Inducible genetic marking complements previous stem cell studies using label retention, which identify quiescent stem cells but give no information on the clonal units maintained by active stem cells. Our observations suggest that the IFE, hair follicle and sebaceous glands contain stem cells, which are independent of bulge stem cells over a one year period. A second surprising observation is that, in contrast to other progenitor cells, such, as O2A oligodendrocyte precursors, which exhibit symmetric proliferation and differentiation, adult epidermal TA cells exhibit both symmetric and asymmetric cell division. This gives rise to a pattern of clone expansion that generates odd numbers of cells in numerous TA derived clones, and explains the slow rate of TA clone expansion (FIG. 2C).

This system finds application in modelling the earliest-stages of cancer development.

### EXAMPLE 3

### Clonal Modelling of Cancer

Cancer is hypothesised to evolve from oncogenic mutation in individual progenitor cells in a background of wild type cells. The mouse model system described above is ideally suited to test this hypothesis in the epidermis and analyse the changes to clonal behaviour that occur during tumour development.

### Development of a Clonal Model of Basal Cell Carcinoma

Basal cell carcinoma (BSC) is the commonest career in Caucasians in the western world. It can cause significant morbidity through local invasion at the tumour site and requires treatment with surgery, cryoablation or radiotherapy. BCC development is linked strongly to over activation of the Hedgehog (HH) signalling pathway. HH ligands, including Sonic Hedgehog (SHH) bind to a transmembrane receptor Patched (PTCH, 1 on FIG. 9). Ligand binding relieves the inhibition of a second transmembrane protein, Smoothened (SMO), by Patched (2). Derepression of Smoothened leads to Gli transcription factors, held in the cytoplasm by a multiprotein complex that includes fused (FU) and suppressor of fused (SuFu,3), translocating to the nucleus (4) where they activate HH target genes (5). Direct transcriptional targets of the HH pathway include Gli1, which is induced by Gli2; Gli1 is expressed at very high levels in sporadic BCC, indicating activation of the HH pathway.

Studies of Gorlin syndrome, a genetic disorder in which sufferers develop multiple BCCs at a young age, revealed mutations in the HH receptor, PTCH. PTCH mutations are also common in sporadic BCC's, where mutations of PTCH and loss of heterozygosity at the PTCH locus are found in 50-70% of cases. Activating mutations in SMO are also found in sporadic BCC. Constitutive activation of the HH pathway in mouse epidermis, by overexpression of either (SHH), activated mutant SMO, Gli1 or Gli2 results in BCC formation. Gli2 expression has been shown to be required for maintaining basal cell carcinomas in mouse skin when expressed from a tetracycline regulated promoter; tumours were induced when Gli2 was overexpressed and regressed when Gli2 was down regulated.

The inducible targeting system we have developed is ideally suited for studying cancer development from its earliest stages, enabling oncogenic mutations to be induced at low frequency in adult mouse epidermis and the progeny of the mutant cells to be followed over an extended time course. Key requirements for this system are

No background recombination prior to induction. We have shown tins is the case for with the AhcreERT mice, in particular in tail and back epidermis.

Reporting of recombinant clones. We have demonstrated EYFP is an effective reporter, suitable for clonal marking. The expression of reporter must be directly linked to the oncogenic event, as ere recombinase is expressed transiently and at low level in Ahcre ERT mice, and the efficiency of ere varies between different alleles.

A single copy of the floxed transgene, to ensure all recombinant clones express the same level of the oncogenic protein.

The oncogenic mutation must generate epidermal tumours with high frequency and short latency in a 129xC57BL/6 background.

To engineer a clonal model of BCC that meets these requirements, we propose the construct shown in FIG. 10 to generate transgenic mice.

The 5 kb keratin 5 promoter gives high level transgene expression restricted to the basal layer of the epidermis and other stratified squamous epithelia. No transgene mRNA is expressed until the stop cassette, flanked by lox p sites (black triangles in FIG. 10) is excised by ere recombinase. In this example, overexpression of Gli2 has been chosen as the model oncogenic event. Gli2 expressed from a keratin 5 promoter gives BCC within 6 months, mostly in the tail epidermis, which is ideal for wholemount imaging. Reporting is by an IRES sequence to create a bicistronic mRNA including EYFP; which ensures stop cassette excision is directly linked to EYFP expression.

The conditional GM2-EYFP (K5G2Y) cassette is targeted by homologous recombination to the hprt locus in mouse embryonic stem cells, which are then used to generate transgenic mice. This approach is well characterised and ensures a single copy of the transgeneis integrated into a constitutively transcribed locus. Animals are bred to homozygosity to generate the hprt mice, which are then crossed onto the Ahcre^{ERT} strain to generate heterozygous Ahcre^{ERT}hprt^{K5G2Y/wt} mice which are used for experiments at 6-8 weeks of age.

As a control we generate the mouse shown in FIG. 11.

The control mouse enables us to determine the initial number of recombinants induced at the hprt locus, and determine if the number, size or differentiation of Gli2overexpressing clones differs from controls in at a given time point.

Titration of Induction at the hprt Locus

Titrate B-NF and Tamoxifen doses to determine the optimum doses to give a frequency of recombination in tail and back skin suitable for resolving individual clones. Recombination frequency is proportional to drug doses used for the AhcreERT mice.

### Effects of Gli2 Expression on Clonal Proliferation and Differentiation

Cohorts of 3 experimental and control mice per time point are induced and sacrificed at 2 days post recombination, weekly up to 6 weeks and monthly thereafter, and clonal fate analysed in wholemounts of tail epidermis and eryosections of dorsal epidermis, analysing clone size, proliferation and differentiation by confocal microscopy and immunostaining as described above, and the frequency of clones which differ from those seen in induced control animals at the corresponding time point are determined. Unlabelled epidermis adjacent to the Gli2 clones is also examined for alterations in proliferation or differentiation. In particular we examine the proportion of asymmetric divisions in labelled clones. Animals which develop tumours are sacrificed and the epidermis analysed as above. These experiments yield a quantitative analysis of clonal fate following Gli2 overexpression; this data is examined to determine if a subset of expanded clones, some of which will go on to form tumours emerges, and determine the time course over which this happens.

### Characterisation of Clonal Phenotypes In Vitro

The power of the system of the invention lies in the fact that all clones within an animal are synchronously induced to express the same level of Gli2; any differences between clones are therefore due to additional oncogenic events. Once the time points at which expanded clones begin to appear have been defined, we will dissect out individual clones from tail epidermis under a fluorescent dissecting microscope, disaggregate the cells and place them in culture, using a modified Rheinwald and Green culture system (Blanpain et al. 2004 Cell vol 118 pp 635-48). Once established, cultures will be purified to remove unlabelled cells by flow cytometry.

In BCC arising from mice in which Gli2 is overexpressed throughout the basal epidermal layer, the tumours that emerge are dependent on continued Gli2 overexpression. We will determine whether cultured clone cells remain dependent on Gli2, by infecting cultured cells with retroviral vectors encoding control and EYFP silencing short hairpin RNA's, thus knocking down expression of the transgenic Gli2without affecting endogenous Gli2 expression.

### Analysis of Molecular Phenotype of Expanded Clones.

Cultured clones are analysed by combined array CGH and expression microarray analysis to determine if there are chromosomal events that accompany altered clonal behaviour. If genes are found to be lost with high frequency, we examine the effects of repairing these defects by infecting cultured cells with retroviruses encoding the deleted gene; likewise we will assay the effect of retroviral knockdown of genes which are overexpressed.

### Effects of Low Dose UV-Irradiation on Gli2Expressing Clones

Low dose UV irradiation enhances-tumor development in Patched heterozygote mice and has been shown to induce p53 mutations in mouse epidermis. We investigate whether such irradiation accelerates the development of Gli2 positive clones into tumours.

Comparison of Molecular Changes in Preneoplastic Clones with Sun Exposed Human Epidermis

Basal cell carcinomas arise within sun exposed human epidermis. We examine the expression of proteins encoded by the human homologues of genes whose expression is altered in gli2 positive clones by immunohistochemistry, using commercially available antibodies where available, to determine if these proteins have a clonal pattern of expression within human epidermis. Where such antibodies are not available we carry out in situ hybridisation for the gene concerned to determine if its expression pattern suggests a clonal lesion; polyclonal antisera are raised against those proteins found to have such a pattern of expression.

### EXAMPLE 4

### Evaluation of Cancer Preventative Agents

The effects of candidate cancer preventative agents on the evolution of Gli2 overexpressing clones is examined by topical treatment tail and back skin of induced mice. Agents to be tested will include retinoids. Clinical studies suggest that whilst retinol and isotretinoin are of no benefit in chemoprevention of BCC and squamous carcinoma (SCC), oral acitretin may inhibit SCC and BCC development in renal transplant patients and topical treatment with the RAR beta and gamma selective retinoid tazarotene results in the regression of BCC. Studies in irradiated patched heterozygote mice show topical tazarotene results in a substantial decrease in the number of BCC induced by UV or ionizing radiation. Experiments with all trans retinoic acid (ATRA) applied topically to tail skin in induced Ahcre^{ERT} R26^{EYFP/wt} indicate that retinoid treatment causes rapid depletion of transit amplifying cell clones and expansion of stem cell clones compared with vehicle only control, indicating topical treatments can alter clonal fate.

We evaluate the effects of topically applied tazarotene and acitretin on the size and number of Gli2 positive clones, to determine the effects of treatment at early timepoints after induction on clonal development and the ability of the drugs to block the development of BCC in clones at later time points. We also investigate the effects of ATRA in view of its inhibition of Gli1 in cultured keratinocytes. Similar studies will be carried out on other classes of chemopreventative agent, such as α-difluoromethylornithine and epidermal growth factor receptor tyrosine kinase inhibitors, eg AG1478 (Tang et al. 2004 J Clin Invest 113(6): 867-75; El-Abaseri et al. 2005 Cancer Res 65(9): 3958-65).

Thus the invention finds application in study of clonal evolution of basal cell carcinoma and provides a model for this disease that will assist in the development of chemopreventative treatment for sun damaged skin.

### EXAMPLE 5

### Regulation of Assymmetric Fate and Cancer Development in Epidermal Progenitor Cells

Our previous work shows that asymmetric cell division is frequent in epidermal progenitor cells, and is associated with asymmetric localisation of numb protein. In vitro work indicates that asymmetric division is significantly less frequent in primary human keratinocytes plated at low density in culture than in murine epidermis, suggesting asymmetrical division is controlled by signals from other cells or the external microenvironment. We wish to determine how asymmetrical fate regulation is regulated and whether loss of this regulation contributes to the development of cancer. Clonal targeting, which enables asymmetric divisions to be visualised and quantitated over time, is an ideal system for this experiment.

An essential component of regulator of asymmetric cell division is the protein numb. Numb is thought to function by localising in only one of two dividing cells, suppressing Notch signalling in the cell in which it accumulates with the result that the cells adopt different fates. Numb regulates the fate of muscle and nerve progenitors. We have demonstrated that numb protein is partitioned asymmetrically between some dividing progenitor cells in murine epidermis (FIG. 8). Abolition of numb function in mice requires the deletion of 2 genes, numb and numb like, and so is not a preferred strategy for a clonal experiment. An alternative means to study numb function is to delete components of the Notch signalling pathway. Conditional deletion of Notch 1 in murine epidermis results in an early hyperproliferative phenotype in which differentiation is disrupted, and the development of squamous carcinoma after a latent period of 15 weeks, when mice were treated with chemical carcinogens dimethylbenzanthracene (DMBA) and tetraphorbol acetate (TPA). Intriguingly, one consequence of the loss of Notch combined with carcinogen exposure was the development of basal cell carcinoma type lesions, expressing high levels of Gli2. This parallels the loss of Notch expression seen in human BCC's; taken together, these results suggest loss of Notch may contribute to the development of BCC.

Unfortunately, existing floxed notch 1 mice do not contain a reporter that is expressed following Notch deletion, so are unsuitable for a clonal experiment to look at the interaction between HH activation and loss of Notch. To study the effects of blocking Notch function in a clonal manner we use a dominant negative mastermind like 1 (Maml-1). Maml-1 is required for Notch mediated transcription, forming a complex with the notch cytoplasmic domain and its coactivator, suppressor of hairless; dominant negative MAML1 mutants block transcription mediated by all 4 of the mammalian Notch receptors. Analysis of Maml-1 dominant negative mutants has shown that a 62 amino acid region of the N terminal part of Maml-1, fused in frame to EGFP (MDN-EGFP) functions to blocks Notch signalling efficiently in both transiently transfected cell lines in vitro and retrovirally transduced primary haemopoietic cell lines in vivo. The availability of a validated fluorescent dominant negative mastermind protein makes this an attractive choice as a reagent to manipulate epidermal progenitor cell fate.

To examine the effects of Notch inactivation on epidermal progenitor fate in a clonal experiment we will generate transgenic mice in which the construct of FIG. 12 is targeted to the HPRT mice. This is designed to fulfill the requirements for a clonal transgenic construct as set out for the Gli2 transgenic above.

We teach constructing MDN ECFP (MDNC), identical to the published MDN EGFP except that a cyan instead of green enhanced fluorescent protein is used. This modification enables double reporting of recombination should the mice be crossed onto other strains in which EYFP has been used as a reporter in double transgenic experiments with the Gli2-EYFP. The MDN ECFP protein will be tested for its ability to block activation of a notch induced reporter in vitro and recapitulate the effects of loss of mastermind in vivo in Xenopus embryos. The construct is targeted to the HPRT locus in the same manner as the Gli2 transgenic mice-described above, to generate hprt^{K5MDNC/K5MDMC} mice (Bronson et al. 1996 Proc Natl Acad Sci USA 93(17): 9067-72). These animals are crossed with homozygous Ahcre^{ERT} mice to generate heterozygous Ahcre^{ERT}hprt^{K5MDNC/wt} animals which are used for the experiments below.

### The Effects of Blocking Notch Signalling on Asymmetric Cell Division, Proliferation and Differentiation of Epidermal Progenitor Cell Clones.

Cohorts of mice are induced with B-NF and tamoxifen, using doses previously defined for the Gli2 transgenic animals. The effects of MDNC overexpression on the number, size and differentiation of clones over a time course following induction is analysed. Staining for Ki67 is used to define the number of 2 cell clones undergoing asymmetric division within the basal layer, as described above. Clones at late timepoints (6 months and 1 year) are analysed to determine if they show evidence of expansion or altered differentiation consistent with premalignant change. Abnormal clones are analysed by culture and by molecular analysis as described for the Gli2 mice (page 10, sections b and c). We also assess the effects of UV irradiation on MDNC expressing clones, as described for the Gli2 animals.

### Effects of Notch Inactivation on Cancer Development in Gli2 Transgenics

The observation that loss of Notch 1 contributes to the development of BCC like tumours suggests that loss of Notch may interact will activated hedgehog signalling in BCC development. To test if this is the case we cross Ahcre^{ERT} hprt^{K5G2Y/wt} transgenic animals, with the Ahcre^{ERT}hprt^{K5MDNC/wt} strain. We test whether the progeny, comprising Ahcre ^{ERT}hprt^{K5MDNC/K5G2Y}, Ahcre^{ERT}hprt^{K5MDNC/wt}, Ahcre^{ERT} hprt^{K5G2Y/wt} and Ahcre^{ERT}hprt^{K5MDNC/K5G2Y} genotypes differ in the incidence of tumour development when induced. Clones are analyse at different time points, and clone cells cultured and analysed as described above.

### Analysis of Human Sunexposed Skin for the Presence of Clones Showing Clonal Loss of Notch Signalling.

Human sun exposed epidermis is screened for evidence of clones showing changes in protein expression that accompany loss of Notch signalling, defined in the experiments above. Antibodies to these proteins are raised.

The experiments described here offer comprehensive insight into the development of non melanoma skin cancers from single cells to overt tumours. By defining the preneoplastic changes in cancer development, new targets and means of early diagnosis can be developed, not only for skin cancer but also for tumours in other squamous epithelia.

All publications mentioned in the above specification are herein incorporated by reference. Various, modifications and variations of the described methods and system of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method of imaging a clonal cell line comprising
(i) providing a test animal comprising a marker gene,
(ii) inducing inheritable activation of said marker in at least one cell of said test animal, wherein inheritable activation is induced in fewer than 1 in 27 cells in the tissue of interest,
(iii) incubating the test animal, and
(iv) visualising those clonal cells which express the marker gene as a result of the inheritable activation.

2. A method according to claim 1 wherein the tissue is epidermis.

3. A method according to claim 1 wherein the visualisation is by confocal microscopy.

4. A method according to claim 1 wherein the visualisation is by wholemount confocal microscopy.

5. A method according to claim 1 wherein inducing inheritable activation is performed by inducing recombination in order to produce expression of said marker.

6. A method according to claim 5 wherein the recombination is induced by administration of B-napthoflavone and tamoxifen.

7. A method according to claim 1 wherein the marker is enhanced yellow fluorescent protein.

8. A method according to claim 1 wherein the recombination system is based on cre-lox.

9. A method according to claim 1 wherein the mouse is AhcreER^{T} and the induction of recombination is carried out by administration of B-napthoflavone together with tamoxifen.

10. A method of assessing the toxicity of a substance or composition comprising imaging according to claim 1 a clonal cell line which has been incubated in the presence of said compound or composition.

11. A method of assessing the carcinogenicity of a substance or composition comprising imaging according claim 1 a clonal cell line which has been incubated in the presence of said compound or composition.

12. A method according to claim 10 or claim 11 further comprising comparing the images of the clonal cell line incubated in the presence of said substance or composition with the characteristics of a corresponding clonal cell line which has not been incubated in the presence of said substance or composition.

13. The method of claim 1 wherein said animal is a mouse comprising AhcreER^{T} and said mouse is used in the monitoring of clonal cell lines.

14. The method of claim 1 wherein said animal is a mouse comprising AhcreER^{T} and said mouse is used in the monitoring of expansion or differentiation of at least one cell arising from a single somatic recombination event.

15. The method according to claim 14 wherein a single clonal cell line is monitored.

16. A method of using a cohort of mice to monitor clonal cells for inheritable activation, wherein at least one single recombination event is induced in each mouse of the cohort at a starting time point, wherein cells in a first mouse of the cohort are examined at a first time point, and cells in a second or further mouse of the cohort are examined at second or further time points thereafter.

17. The method according to claim 16 wherein said mouse comprises AhcreER^{T}.

18. The method of claim 13 wherein said mouse further comprises R26^{EYFP/EYFP}.
